SEQUENCE LISTING (37 sequences)

SEQ ID NO: 1 - DNA, 1464 bp, Streptococcus gallolyticus UCN34
atgaaaaaagcattagttggatctttagcgaccctaactgtcgttgccggtctcgcatcagctcaaggggtaaaagctgacgaaatcgtttcaggacagacttacaaaatcactgcaaaacatagtggtaaagctcttgacgttgcatcaaaagcaacttacgcaggagctaacgtgcaacaatggtcttataacggcggcactaaccaacaatggaaagttgttgatacaggagacggatactacaaattagtatcagtaaacagtggtaaagtgctagacgtagcaagtgcagcaacttacaatggtgctaacgtacaacaatgggacgatactaacggaacatgccaacgctggaaaatcgtagatactggtgacggttcttacaaactaatctctgctgtcagcggtaaagctcttgacgttgcagcaaactctagcgatgacggtgccaacgtacaacaatgggatgacaacggtggttggaaccaaaaatgggaattcacaaaagtaagtgacagtggaaacactggtggtgatactggtggcgacactactactccagtttcaactagtggtttctacacaaacggtactacactttatgacgcaaaaggtaatccatttgtgatgcgcggtatcaaccacgctcactcttggtaccaaggttatgaagatactgctattcctgccattgcaaaaactggcgctaactgtgttcgcttagttatcacagatggtcaacaatacagtaaaacttcactctctgaacttcaaaaacttattcaactttgtaaagataacaaattaattgctatcgttgaagtgcatgacgctactggttcagataacattcaagacttagaaaaagctgctgattactggattgaaatgaagagtgctttgattggaaacgaagcaaacgtcatccttaacatcgctaacgaatggggcggtgcttgggacagttcaaactgggcagcaggttaccaacaagttattccaaaacttcgtaatgctggtatcaaaaacacacttatggtcgattgtgctggttggggacaatacccacaatctatcgcagaagcaggtcaaagtgtcgttcaagctgattcacaacacaacatcatgttctctattcacatgtacgaatatgctggtgcatacggcgcagttcaatcaaacattgactctgcacttaacgtaggtgtaccactttgtatcggtgaattcggtatccgacacactaacggcgatgtagattacaagacaatcatgagctactctcaatctaagggagttggttacctcggttggtcttggaaaggtaacggcgacacttgggcttacctcgatatcgcacaagattggagcggttcacaattaactgaacaaggtaacgctattatctatgaccaaaatggtatcaagaacacatctaaacgttgtacaatttttgactaa

SEQ ID NO: 2 - protein, 487 aa, Streptococcus gallolyticus UCN34
MKKALVGSLATLTVVAGLASAQGVKADEIVSGQTYKITAKHSGKALDVASKATYAGANVQQWSYNGGTNQQWKVVDTGDGYYKLVSVNSGKVLDVASAATYNGANVQQWDDTNGTCQRWKIVDTGDGSYKLISAVSGKALDVAANSSDDGANVQQWDDNGGWNQKWEFTKVSDSGNTGGDTGGDTTTPVSTSGFYTNGTTLYDAKGNPFVMRGINHAHSWYQGYEDTAIPAIAKTGANCVRLVITDGQQYSKTSLSELQKLIQLCKDNKLIAIVEVHDATGSDNIQDLEKAADYWIEMKSALIGNEANVILNIANEWGGAWDSSNWAAGYQQVIPKLRNAGIKNTLMVDCAGWGQYPQSIAEAGQSVVQADSQHNIMFSIHMYEYAGAYGAVQSNIDSALNVGVPLCIGEFGIRHTNGDVDYKTIMSYSQSKGVGYLGWSWKGNGDTWAYLDIAQDWSGSQLTEQGNAIIYDQNGIKNTSKRCTIFD

SEQ ID NO: 3 - protein, 466 aa, Streptococcus gallolyticus UCN34
QGVKADEIVSGQTYKITAKHSGKALDVASKATYAGANVQQWSYNGGTNQQWKVVDTGDGYYKLVSVNSGKVLDVASAATYNGANVQQWDDTNGTCQRWKIVDTGDGSYKLISAVSGKALDVAANSSDDGANVQQWDDNGGWNQKWEFTKVSDSGNTGGDTGGDTTTPVSTSGFYTNGTTLYDAKGNPFVMRGINHAHSWYQGYEDTAIPAIAKTGANCVRLVITDGQQYSKTSLSELQKLIQLCKDNKLIAIVEVHDATGSDNIQDLEKAADYWIEMKSALIGNEANVILNIANEWGGAWDSSNWAAGYQQVIPKLRNAGIKNTLMVDCAGWGQYPQSIAEAGQSVVQADSQHNIMFSIHMYEYAGAYGAVQSNIDSALNVGVPLCIGEFGIRHTNGDVDYKTIMSYSQSKGVGYLGWSWKGNGDTWAYLDIAQDWSGSQLTEQGNAIIYDQNGIKNTSKRCTIFD

SEQ ID NO: 4 - protein, 307 aa, Xanthomonas campestris
GLSVSGTQLKESNGNTLILRGINLPHAWFADRTDAALAQIAATGANSVRVVLSSGHRWNRTPEAEVARIIARCKALGLIAVLEVHDTTGYGEDGAAGSLANAASYWTSVRTALVGQEDYVIINIGNEPFGNQLSASEWVNGHANAIATLRGAGLTHALMVDAPNWGQDWQFYMRDNAAALLARDSRRNLIFSVHMYEVFGSDAVVDSYLRTFRSNNLALVVGEFGADHRGAPVDEAAIMRRAREYGVGYLGWSWSGNDSSTQSLDIVLGWDPARLSSWGRSLIQGPDGIAATSRRARVFGARVRAME

SEQ ID NO: 5 - protein, 351 aa, Unknown
  Streptomyces sp.
AEAATGIRVGNGRVYEANGNEFVMRGVNHAHAWYPNRTGSIAHIKAKGANTVRVVLANGDRWTRTSASEVSSIIGQCKQNRLICVLEVHDTTGYGEDGAATSLSRAADYWIGVKSALEGQENYVVINIGNEPFGNNGYDRWTSDTIAAVQKLRNAGFDHALMVDAPNWGQDWSNTMRNNASTVFNSDPDRNTIFSIHMYGVYNTASEVQSYLNHFVGNRLPIVVGEFGHNHGDGDPDENAIMATAQSLRVGYLGWSWSGNGGGVEYLDMVNGFDPNSLTGWGQRFFNGANGISATSREATVYGGGSGGGSGGTAPNGYPYCVDGSASDPDGDGWGWENQRSCVVRGSAADG

SEQ ID NO: 6 - DNA, 1494 bp, Artificial Sequence
  Synthetic construct
gtgagaagcaaaaaattgtggatcagcttgttgtttgcgttaacgttaatctttacgatggcgttcagcaacatgagcgcgcaggctgctggaaaacaaggcgttaaagcagatgaaattgtttcaggccagacgtataaaatcacagcaaaacattcaggcaaagcactggatgttgcaagcaaagcaacatatgcaggcgcaaatgttcaacagtggtcatataatggcggaacaaatcaacaatggaaagtcgttgatacaggcgacggctattacaaactggtttcagttaatagcggcaaagttctggatgtcgcatcagcagcaacatacaatggcgctaatgtccaacaatgggatgatacaaatggcacatgccagagatggaaaattgtcgatacaggtgatggcagctataaactgatttcagcagttagcggaaaagcgcttgatgttgcggcaaattcatcagatgatggcgcaaacgtgcagcagtgggatgacaatggcggatggaatcaaaaatgggaatttacgaaagttagcgactcaggcaatacaggcggagatacgggtggcgatacaacaacaccggtttcaacatcaggcttttatacaaacggaacgacactgtatgatgcaaaaggcaatccgtttgttatgcgtggcattaatcatgcacattcatggtatcaaggctatgaagatacagcaattccggctattgcaaaaacaggcgctaattgcgttagactggttattacagatggccagcaatattcaaaaacatcactgagcgaactgcagaaactgattcaactgtgcaaagacaacaaactgatcgcaattgttgaagttcatgatgctacaggcagcgataatattcaggatctggaaaaagcagcggactattggattgaaatgaaatcagcactgattggcaatgaagcgaatgtcattctgaacattgcaaatgaatggggaggcgcatgggattcatcaaattgggcagcaggctatcaacaagttattccgaaactgagaaatgcaggcattaaaaacacacttatggttgattgcgcaggctggggacaatatccgcaatcaattgcagaagcaggccaaagcgttgttcaagcagattcacaacataacatcatgtttagcatccatatgtacgaatacgcaggcgcatatggcgcagttcaatcaaatattgatagcgcactgaatgttggcgttccgctgtgcattggcgaatttggcattagacatacgaatggtgatgtcgactataaaacgatcatgagctattcacaatcaaaaggcgttggctatctgggctggtcatggaaaggcaacggcgatacatgggcatatctggatattgcacaagattggtcaggctcacaactgacagaacaaggcaatgcaattatctatgatcagaacggcatcaaaaatacgtcaaaacgctgcacgatctttgat

SEQ ID NO: 7 - protein, 498 aa, Artificial Sequence
  protein expressed from synthetic construct
MRSKKLWISLLFALTLIFTMAFSNMSAQAAGKQGVKADEIVSGQTYKITAKHSGKALDVASKATYAGANVQQWSYNGGTNQQWKVVDTGDGYYKLVSVNSGKVLDVASAATYNGANVQQWDDTNGTCQRWKIVDTGDGSYKLISAVSGKALDVAANSSDDGANVQQWDDNGGWNQKWEFTKVSDSGNTGGDTGGDTTTPVSTSGFYTNGTTLYDAKGNPFVMRGINHAHSWYQGYEDTAIPAIAKTGANCVRLVITDGQQYSKTSLSELQKLIQLCKDNKLIAIVEVHDATGSDNIQDLEKAADYWIEMKSALIGNEANVILNIANEWGGAWDSSNWAAGYQQVIPKLRNAGIKNTLMVDCAGWGQYPQSIAEAGQSVVQADSQHNIMFSIHMYEYAGAYGAVQSNIDSALNVGVPLCIGEFGIRHTNGDVDYKTIMSYSQSKGVGYLGWSWKGNGDTWAYLDIAQDWSGSQLTEQGNAIIYDQNGIKNTSKRCTIFD

SEQ ID NO: 8 - protein, 469 aa, Artificial Sequence
  protein expressed from synthetic construct
AGKQGVKADEIVSGQTYKITAKHSGKALDVASKATYAGANVQQWSYNGGTNQQWKVVDTGDGYYKLVSVNSGKVLDVASAATYNGANVQQWDDTNGTCQRWKIVDTGDGSYKLISAVSGKALDVAANSSDDGANVQQWDDNGGWNQKWEFTKVSDSGNTGGDTGGDTTTPVSTSGFYTNGTTLYDAKGNPFVMRGINHAHSWYQGYEDTAIPAIAKTGANCVRLVITDGQQYSKTSLSELQKLIQLCKDNKLIAIVEVHDATGSDNIQDLEKAADYWIEMKSALIGNEANVILNIANEWGGAWDSSNWAAGYQQVIPKLRNAGIKNTLMVDCAGWGQYPQSIAEAGQSVVQADSQHNIMFSIHMYEYAGAYGAVQSNIDSALNVGVPLCIGEFGIRHTNGDVDYKTIMSYSQSKGVGYLGWSWKGNGDTWAYLDIAQDWSGSQLTEQGNAIIYDQNGIKNTSKRCTIFD

SEQ ID NO: 9 - protein, 29 aa, Bacillus subtilis
MRSKKLWISLLFALTLIFTMAFSNMSAQA

SEQ ID NO: 10 - protein, 32 aa, Trichoderma reesei
MVSFTSLLAASPPSRASCRPAAEVESVAVEKR

SEQ ID NO: 11 - protein, 16 aa, Trichoderma reesei
MKANVILCLLAPLVAA

SEQ ID NO: 12 - protein, 19 aa, Trichoderma reesei
MRYRTAAALALATGPFARA

SEQ ID NO: 13 - protein, 18 aa, Trichoderma reesei
MIVGILTTLATLATLAAS

SEQ ID NO: 14 - protein, 17 aa, Trichoderma reesei
MYRKLAVISAFLATARA

SEQ ID NO: 15 - protein, 23 aa, Fusarium verticillioides
MLLNLQVAASALSLSLLGGLAEA

SEQ ID NO: 16 - protein, 19 aa, Fusarium verticillioides
MKLNWVAAALSIGAAGTDS

SEQ ID NO: 17 - protein, 19 aa, Fusarium verticillioides
MASIRSVLVSGLLAAGVNA

SEQ ID NO: 18 - protein, 22 aa, Fusarium verticillioides
MWLTSPLLFASTLLGLTGVALA

SEQ ID NO: 19 - protein, 16 aa, Fusarium verticillioides
MRFSWLLCPLLAMGSA

SEQ ID NO: 20 - protein, 22 aa, Fusarium verticillioides
MRLLSFPSHLLVAFLTLKEASS

SEQ ID NO: 21 - protein, 20 aa, Fusarium verticillioides
MQLKFLSSALLLSLTGNCAA

SEQ ID NO: 22 - protein, 18 aa, Fusarium verticillioides
MKVYWLVAWATSLTPALA

SEQ ID NO: 23 - protein, 19 aa, Fusarium verticillioides
MVRFSSILAAAACFVAVES

SEQ ID NO: 24 - protein, 20 aa, Podospora anserine
MIHLKPALAALLALSTQCVA

SEQ ID NO: 25 - protein, 17 aa, Podospora anserine
MALQTFFLLAAAMLANA

SEQ ID NO: 26 - protein, 19 aa, Podospora anserine
MKLNKPFLAIYLAFNLAEA

SEQ ID NO: 27 - protein, 20 aa, Chaetomium globosum
MAPLSLRALSLLALTGAAAA

SEQ ID NO: 28 - protein, 19 aa, Thermoascus aurantiacus
MVRPTILLTSLLLAPFAAA

SEQ ID NO: 29 - protein, 21 aa, Aspergillus terreus
MHMHSLVAALAAGTLPLLASA

SEQ ID NO: 30 - protein, 19 aa, Aspergillus fumigatus
MVHLSSLAAALAALPLVYG

SEQ ID NO: 31 - protein, 17 aa, Aspergillus fumigatus
MRFSLAATTLLAGLATA

SEQ ID NO: 32 - protein, 19 aa, Aspergillus fumigatus
MVVLSKLVSSILFASLVSA

SEQ ID NO: 33 - protein, 19 aa, Aspergillus kawachii
MVQIKAAALAMLFASHVLS

SEQ ID NO: 34 - protein, 17 aa, Magnaporthe grisea
MKASSVLLGLAPLAALA

SEQ ID NO: 35 - protein, 19 aa, Saccharomyces cerevisiae
MRFPSIFTAVLFAASSALA

SEQ ID NO: 36 - protein, 85 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVSLDKR

SEQ ID NO: 37 - protein, 20 aa, Saccharomyces cerevisiae
MLLQAFLFLLAGFAAKISAR

(19) United States
(12) Patent Application Publication
(10) Pub. No.: US 2017/0211053 A1
(43) Pub. Date: Jul. 27, 2017

(54) COMPOSITIONS COMPRISING BETA MANNANASE AND METHODS OF USE

(71) Applicant: Danisco US Inc., Palo Alto, CA (US)

(72) Inventors: Steven Le, Palo Alto, CA (US); Zhen Qian, Palo Alto, CA (US)

(21) Appl. No.: 15/514,750

(22) PCT Filed: Sep. 30, 2015
(86) PCT No.: PCT/US2015/053155

§ 371 Date: Mar. 27, 2017

(30) Foreign Application Priority Data
Sep. 30, 2014 (CN) PCT/CN2014/087867

(51) Int. Cl. C12N 9/24; C12P 19/02; C12P 19/14

(52) U.S. Cl. CPC C12N 9/2491; C12P 19/14; C12Y 302/01025; C12P 19/02

(57) ABSTRACT

The present compositions and methods relate to a beta-mannanase from Streptococcus gallolyticus, polynucleotides encoding the beta-mannanase, and methods of make and/or use thereof. Formulations containing the beta-mannanase are suitable for use in hydrolyzing lignocellulosic biomass substrates, especially those comprising a measurable level of galactoglucomannan (GGM) and/or glucomannan (GM).

### CROSS-REFERENCE TO RELATED APPLICATIONS

This application claims the benefit of priority from PCT Application No. PCT/CN2014/087867, filed in the China Intellectual Property Office on Sep. 30, 2014, the entirety of which is herein incorporated by reference.

### FIELD OF THE INVENTION

The present compositions and methods relates to a beta-mannanase derived from Streptococcus gallolyticus, polynucleotides encoding the beta-mannanase, and methods for the production and use thereof. Formulations containing the recombinant beta-mannanase have a wide variety of uses, for instance, in hydrolyzing certain soft-wood type lignocellulosic materials and/or lignocellulosic biomass substrates comprising galactoglucomannan (GGM) and/or glucomannan (GM).

### REFERENCE TO SEQUENCE LISTING SUBMITTED ELECTRONICALLY

The content of the electronically submitted sequence listing in ASCII text file (Name: 20150930_NB40792WOPCT2_SequenceListing_ST25.txt; Size: 34,824 bytes, and Date of Creation: Sep. 29, 2015) filed with the application is incorporated herein by reference in its entirety.

### BACKGROUND

Cellulose and hemicellulose are the most abundant plant materials produced by photosynthesis. They can be degraded and used as an energy source by numerous microorganisms (e.g., bacteria, yeast and fungi) that produce extracellular enzymes capable of hydrolysis of the polymeric substrates to monomeric sugars (Aro et al., (2001) J. Biol. Chem., 276: 24309-24314). As the limits of non-renewable resources approach, the potential of cellulose to become a major renewable energy resource is enormous (Krishna et al., (2001) Bioresource Tech., 77: 193-196). The effective utilization of cellulose through biological processes is one approach to overcoming the shortage of foods, feeds, and fuels (Ohmiya et al., (1997) Biotechnol. Gen. Engineer Rev., 14: 365-414).

Most of the enzymatic hydrolysis of lignocellulosic biomass materials focus on cellulases, which are enzymes that hydrolyze cellulose (comprising beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”) (Knowles et al., (1987) TIBTECH 5: 255-261; and Schulein, (1988) Methods Enzymol., 160: 234-243). Endoglucanases act mainly on the amorphous parts of the cellulose fiber, whereas cellobiohydrolases are also able to degrade crystalline cellulose (Nevalainen and Penttila, (1995) Mycota, 303-319). Thus, the presence of a cellobiohydrolase in a cellulase system is required for efficient solubilization of crystalline cellulose (Suurnakki et al., (2000) Cellulose, 7: 189-209). Beta-glucosidase acts to liberate D-glucose units from cellobiose, cello-oligosaccharides, and other glucosides (Freer, (1993) J. Biol. Chem., 268: 9337-9342).

In order to obtain useful fermentable sugars from lignocellulosic biomass materials, however, the lignin will typically first need to be permeabilized, for example, by various pretreatment methods, and the hemicellulose disrupted to allow access to the cellulose by the cellulases. Hemicelluloses have a complex chemical structure and their main chains are composed of mannans, xylans and galactans. Mannan-type polysaccharides are found in a variety of plants and plant tissues, for example, in seeds, roots, bulbs and tubers of plants. Such saccharides may include mannans, galactomannas and glucomannans, and they typically containing linear and interspersed chains of linear beta-1,4-linked mannose units and/or galactose units. Most types of mannans are not soluble in water, forming the hardness characteristic of certain plant tissues like palm kernels and ivory nuts. Galactomannas, on the other hand, tend to be water soluble and are found in the seed endosperm of leguminous plants, and are thought to help with retention of water in those seeds.

Enzymatic hydrolysis of the complex lignocellulosic structure and rather recalcitrant plant cell walls involves the concerted and/or tandem actions of a number of different endo-acting and exo-acting enzymes (e.g., cellulases and hemicellulases). Beta-xylanases and beta-mannanases are endo-acting enzymes, beta-mannosidase, beta-glucosidase and alpha-galactosidases are exo-acting enzymes. To disrupt the hemicelulose, xylanases together with other accessory proteins (non-limiting examples of which include L-α-arabinofuranosidases, feruloyl and acetylxylan esterases, glucuronidases, and β-xylosidases) can be applied.

Endo-1,4-beta-D-mannanases (E.C. 3.2.1.78) catalyzes the random hydrolysis of beta-1,4-mannosidic linkages in the main chain of mannan, galactomannanan, glucomannan, and galactoglucomannan, releasing short and long-chain oligomannosides. The short-chain oligomannosides may include mannobiose and mannotriose, although sometimes may also include some mannose. These can be further hydrolyzed by beta-mannosidases (E.C.3.2.1.25). In addition, the side-chain sugars of heteropolysaccharides can be further hydrolyzed, for example, to completion, by alpha galactosidase, beta-glucosidase, and/or by acetylmannan esterases. Puls J., (1997) Macromol. Symp. 120:183-196.

Beta-mannanases have been isolated from bacteria, fungi, plants and animals. See, Araujo A. et al., (1990) J. App. Bacteriol. 68:253-261; Dutta S. et al., (1997) Plant Physiol. 113:155-161; Puchar V. et al., (2004) Biochim. Biophys. Acta 1674:239-250. Genes encoding these enzymes from a number of organisms have also been cloned and sequenced, many if not all have been classified also as members of glycosyl hydrolase (GH) family 5 or 26, based on their sequences. See, e.g., Bewley D. J., (1997) Planta 203:454-459; Halstead J. R. et al., (2000) FEMS Microl. Lett. 192:197-203; Xu B. et al., (2002) Eur. I Biochem. 269:1753-1760; Henrissat, B. (1991) Biochem. 1 280:309-316. Although most beta-mannanases are secreted by the organisms from which they are originated, some are known to be associated with the cells. From a given organism there may be more than one mannanases with different isoelectric points derived from different genes or different products of the same genes, which fact is thought to be an indication of the importance of these enzymes.

Beta-mannanases have been used in commercially applications in, for example, industries such as the paper and pulp industry, foodstuff and feed industry, pharmaceutical industry and energy industry. Lee J T., et al., (2003) Poult. Sci. 82:1925-1931; McCutchen M. C., et al., (1996) Biotechnol. Bioeng. 52:332-339; Suurnakki A., et al., (1997) Adv. Biochem. Eng. Biotechnol., 57:261-287. Depending on the microorganisms from which the mannanases are derived, however, different beta-mannanases may have different properties and activity profiles that may make them more suitable for one or more industrial applications but not for others. The hydrolysis of lignocellulosic biomass substrates, especially those from plant sources, is notoriously difficult, accordingly few if any mannanases that have been found to be useful in other industrial applications have been utilized to hydrolyze lignocellulosic materials.

Thus there exists a need to identify mannanases and/or compositions comprising such enzymes that are effective at and capable of, in conjunction with commercial, newly identified, or engineered cellulases and other hemicellulases, converting a wide variety of plant-based and/or other cellulosic or hemicellulosic materials into fermentable sugars with sufficient or improved efficacy, improved fermentable sugar yields, and/or improved capacity to act on a greater variety of cellulosic feedstock. The production of new mannanases using engineered microbes is also important and desirable because these are means through which enzymes can be cost-effectively made.

### SUMMARY OF THE INVENTION

One aspect of the present compositions and methods is the application or use of a highly active beta-mannanase isolated from the bacterial species Streptococcus gallolyticus strain, to hydrolyze a lignocellulosic biomass substrate. The herein described sequence of SEQ ID NO:2 was identified from the genome sequence of Streptococcus gallolyticus strain UCN34 (“SgaMan1” herein), which has been annotated to be a proported mannanase upon completion of genomic sequencing of Streptococcus gallolyticus strain UCN34, and given an accession number of CBI12654.1 at the National Center for Biotechn000gy Information, U.S. National Library of Medicin. To date this sequence has not been recombinantly expressed, expressed in an industrially or commercially relevant amount, or applied in industrial applications. SgaMan1 polypeptides have not been expressed by an engineered microorganism, or coexpressed with, or included in a composition with, one or more cellulase genes and/or one or more hemicellulases.

Therefore an aspect of the present invention is the discovery that polypeptides having at least 55% (e.g., at least 55%, at last 60%, at least 65%, at least 70%, at least 75%, at least 80%, at least 85%, at least 90%, at least 91%, at least 91%, at least 92%, at least 93%, at least 94%, at least 95%, at least 96%, at least 97%, at least 98%, at least 99% or higher) identity to SEQ ID NO:2, or to the mature sequence of SEQ ID NO:3, which is residues 22-487 of SEQ ID NO:2, have beta-mannanase activity. Another aspect of the present invention is the discovery that, when such a polypeptide is combined with one or more cellulases and/or one or more other hemicellulases confer improved capacity of that composition or mixture to hydrolyze of lignocellulosic biomass substrates. Such improvements include, for example, one or more of the properties selected from: an increased glucan conversion, an increased glucose yield from a given biomass substrate, an increased xylan conversion, an increased xylose yield, an increased total soluble sugar yield from a given biomass substrate, a more rapid liquefaction of a given biomass substrate at a solids level, and a more rapid viscosity reduction of a biomass substrate at a solids level. Improvements also may include the surprising finding that such a polypeptide can be used to boost the cellulosic biomass conversion and hydrolysis when in combination with a cellulase mixture or composition, which optionally further comprises one or more other hemicellulase. The resulting mixture comprising the SgaMan1 polypeptide has improved hydrolysis performance as compared to a counterpart mixture having all the other enzymes at the same concentrations/proportion/amounts, but without the SgaMan1. In some embodiments, the SgaMan1 polypeptides can substitute, for example, for up to about 20 wt. % (e.g., up to about 20 wt. %, up to about 18 wt. %, up to about 16 wt. %, up to about 14 wt. %, up to about 12 wt. %, up to about 10 wt. %, up to about 8 wt. %, up to about 5 wt. %, etc) of a cellulase mixture or composition, and the substituted composition when used to hydrolyze a given lignocellulosic biomass substrate will retain its capacity and hydrolysis performance, or even have improved hydrolysis (e.g., higher glucan and/or xylan conversion, higher production of total sugars, faster liquefaction, and/or improved viscosity reduction) than a un-substituted counterpart cellulase mixture or composition of otherwise the same enzyme composition and the same total protein.

An aspect of the present composition and methods pertains to a beta-mannanase polypeptide of cellulose binding protein derived from Streptococcus gallolyticus, or a suitable variant thereof having beta-mannanase activity, referred to herein as “SgaMan1” or a “SgaMan1 polypeptide,” nucleic acids encoding the same, compositions comprising the same, and methods of producing and applying the beta-mannanase polypeptides and compositions comprising thereof in hydrolyzing or converting lignocellulosic biomass into soluble, fermentable sugars. Particularly suitable lignocellulosic biomass materials are those that contain galactoglucomannan (GGM) and/or glucomannan (GM). Such fermentable sugars can then be converted into cellulosic ethanol, fuels, and other biochemicals and useful products. In certain embodiments, the beta-mannanase polypeptides, when combined with an enzyme mixture comprising at least one cellulase or at least one other hemicellulase, or with an enzyme mixture comprising at least one cellulase and at least one other hemicellulase, resulted in an enzyme mixture that is capable of increased or enhanced capacity to hydrolyze a lignocellulosic biomass material, as compared to, for example, other beta-mannanases from various microbes, which have similar pH optimum and/or similar temperature optimum.

Such increased or enhanced capacity to hydrolyze a lignocellulosic biomass material is reflected, for example, in substantially increased production of not only total soluble sugars, but surprisingly also increased production of glucose (reflecting a higher glucan conversion) and/or increased production of xylose (reflecting a higher xylan conversion), produced by enzymatic hydrolysis of a given lignocellulosic biomass substrate pretreated in a certain way.

The increased or enhanced capacity to hydrolyze a lignocellulosic biomass material can also be reflected in the desirable capacity of such an enzyme composition to improve or accelerate liquefaction and/or reduce viscosity of the pretreated biomass material. Such a viscosity/liquefaction benefit is the most prominent if a high solids level of the biomass material is used as a substrate. The viscosity/liquefaction benefits are also substantial and important when the enzyme composition/mixture is used to break down or hydrolyze a woody biomass, which tends to be highly fibrous and recalcitrant, making for particularly viscous feedstocks.

The increased or enhanced capacity to hydrolyze a lignocellulosic biomass allows the substitution of up to about 20 wt. % (e.g., up to about 20 wt. %, up to about 18 wt. %, up to about 16 wt. %, up to about 14 wt. %, up to about 12 wt. %, up to about 10 wt. %, up to about 8 wt. %, up to about 5 wt. %, etc) of any given cellulase composition, which optionally comprises one or more other hemicellulases, with a SgaMan1polypeptide, thereby reducing the amount of cellulase composition and the enzymes therein used to hydrolyze a given substrate without sacrificing performance. Indeed, the hydrolysis performance may even be improved using the substituted composition. Reducing the amount of cellulase composition as well as the amount of enzymes therein required to hydrolyze or saccharify a lignocellulosic biomass results in a substantial cost-savings to produce a cellulosic sugar, which can then be made into ethanol or other down-stream valuable bio-chemicals and useful products.

Aspects of the present compositions and methods are drawn to beta-mannanase derived from Streptococcus gallolyticus, referred to herein as “SgaMan1” or “SgaMan1 polypeptides,” nucleic acids encoding the same, and methods of producing and employing the beta-mannanase in various industrially useful applications, for example, in hydrolyzing or converting lignocellulosic biomass into soluble, fermentable sugars. Such fermentable sugars can then be converted into cellulosic ethanol, fuels, and other bio-chemicals and useful products. As demonstrated herein, SgaMan1 polypeptides as well as compositions comprising SgaMan1 polypeptides have improved performance, when combined with at least one cellulase and/or at least one other hemicellulase, in hydrolyzing lignocellulosic biomass substrates, especially those that contain at least some measurable levels of galactoglucomannan (GGM) and/or glucomannan (GM), as compared to other beta-mannanases from similar microorganisms having similar pH optimums and/or temperature optimums. The improved performance may be that the SgaMan1 polypeptides and/or enzyme compositions comprising SgaMan1 polypeptides produces increased amounts of total soluble sugars when used to hydrolyze a lignocellulosic biomass substrate, under suitable conditions for the enzymatic hydrolysis, when compared to other microbial beta-mannanases having similar pH optimums and/or temperature optimums. Surprisingly the SgaMan1 polypeptides and/or the compositions comprising such polypeptides also have improved glucan conversion and/or improved xylan conversion, as compared to those other microbial beta-mannanases having similar pH optimums and/or temperature optimums. The improved performance may alternatively or also be that the SgaMan1 polypeptides and/or enzyme compositions comprising SgaMan1 polypeptides confer rapid viscosity reduction/liquefaction to the biomass substrate, such that the overall hydrolysis is improved in not only effectiveness but also efficiency.

In some embodiments, a SgaMan1 polypeptide is applied together with, or in the presence of, one or more cellulases in an enzyme composition to hydrolyze or breakdown a suitable biomass substrate. The one or more cellulases may be, for example, one or more beta-glucosidases, cellobiohydrolases, and/or endoglucanases. For example, the enzyme composition may comprise a SgaMan1 polypeptide, a beta-glucosidase, a cellobiohydrolase, and an endoglucanase. In some embodiments, at least one of the cellulases is heterologous to the SgaMan1, in that at least one of the cellulases is not derived from a Streptococcus gallolyticus. In some embodiments, at least two among the cellulases are heterologous from each other.

In some embodiments, a SgaMan1 polypeptide is applied together with, or in the presence of, one or more other hemicellulases in an enzyme composition. The one or more other hemicellulases may be, for example, other mannanases, xylanases, beta-xylosidases, and/or L-arabinofuranosidases. In some embodiments, at least one of the other hemicellulases is heterologous to the SgaMan1, in that at least one of the other hemicellulases, which may be selected from one or more other mannanases, xylanases, beta-xylosidases, and/or L-arabinofuranosidases, is not derived from a Streptococcus gallolyticus. In certain embodiments, at least two of the other hemicellulases are heterologous to each other.

In further embodiments, the SgaMan1 polypeptide is applied together with, or in the presence of, one or more cellulases and one or more other hemicellulases in an enzyme composition. For example, the enzyme composition comprises a SgaMan1 polypeptide, no or one or two other mannanases, one or more cellobiohydrolases, one or more endoglucanases, one or more beta-glucosidases, no or one or more xylanases, no or one or more beta-xylosidases, and no or one or more L-arabinofuranosidases.

In some embodiments, a SgaMan1 polypeptide is used to substitute up to about 20 wt. % (based on total weight of proteins in a composition) (e.g., up to about 20 wt. %, up to about 18 wt. %, up to about 16 wt. %, up to about 14 wt. %, up to about 12 wt. %, up to about 10 wt. %, up to about 8 wt. %, up to about 5 wt. %, etc) of an enzyme composition comprising one or more cellulases, optionally also one or more other non-SgaMan1 hemicellulases. In some embodiments, the thus-substituted enzyme composition has similar or improved saccharification performance as the counterpart unsubstituted enzyme composition having no SgaMan1 present but all the other cellulases and/or hemicellulases, as well as the same total weight of proteins in the composition. In some embodiments, the substituted enzyme composition can produce the same amount of glucose and/or xylose, or an about 5% higher amount of glucose and/or xylose, about 7% higher amount of glucose and/or xylose, about 10% higher amount of glucose and/or xylose, or an even greater amount of glucose and/or xylose from the same lignocellulosic biomass substrate, as compared to the un-substituted counterpart enzyme composition having no SgaMan1 but all the other cellulases and/or hemicellulases, and comprising the same total weight of proteins in the composition. In some embodiments, when used to hydrolyze a given lignocellulosic biomass substrate at a given solids level, the substituted enzyme composition reduces the viscosity of the biomass substrate by the same extent or to a higher extent, when compared to the un-substituted counterpart enzyme composition comprising no SgaMan1 but all the other cellulases and/or hemicellulases, and comprising the same total weight of proteins in the composition.

In certain embodiments, a SgaMan1 polypeptide, or a composition comprising the SgaMan1 polypeptide is applied to a lignocellulosic biomass substrate or a partially hydrolyzed lignocellulosic biomass substrate in the presence of an ethanologen microbe, which is capable of metabolizing the soluble fermentable sugars produced by the enzymatic hydrolysis of the lignocellulosic biomass substrate, and converting such sugars into ethanol, biochemicals or other useful materials. Such a process may be a strictly sequential process whereby the hydrolysis step occurs before the fermentation step. Such a process may, alternatively, be a hybrid process, whereby the hydrolysis step starts first but for a period overlaps the fermentation step, which starts later. Such a process may, in a further alternative, be a simultaneous hydrolysis and fermentation process, whereby the enzymatic hydrolysis of the biomass substrate occurs while the sugars produced from the enzymatic hydrolysis are fermented by the ethanologen.

The SgaMan1 polypeptide, for example, may be a part of an enzyme composition, which is a whole broth product of an engineered microbe capable of expressing or over-expressing such a polypeptide under suitable conditions. In certain embodiments, the SgaMan1 polypeptide may be genetically engineered to express in a bacterial host cell, for example, in Escherichia, Bacillus, Lactobacillus, Pseudomonas, or Streptomyces. In certain embodiments, the SgaMan1 polypeptide may be genetically engineered to express in a fungal host cell, for example, in a host cell of any one of the filamentous forms of the subdivision Eumycotina. Thus suitable filamentous fungal host cells may include, without limitation, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus,Scytaldium, Schizophyllum, Sporotrichum, Talaromyces, Thermoascus, Thielavia, Tolypocladium, Trametes, and Trichoderma.

The engineered microbe expressing or over-expressing the SgaMan1 polypeptide may also express and/or secrete one or more or all of one or more cellulases and optionally also one or more other hemicellulases. The one or more cellulases may be selected from, for example, one or more endoglucanases, one or more beta-glucosidases, and/or one or more cellobiohydrolases. The one or more other hemicellulases may be selected from, for example, one or more other beta-mannanases, one or more Alpha-L-arabinofuranosidases, one or more xylanases, and/or one or more beta-xylosidases. The resulting enzyme mixture comprising the SgaMan1 polypeptide is a “co-expressed enzyme mixture” for the purpose of this application.

In another embodiment, the engineered microbe expressing or over-expressing the SgaMan1 polypetpide may be one that is different from the one or more other microbes expressing one or more of the cellulases and/or one or more of the other hemicellulases. The one or more cellulases may be selected from, for example, one or more endoglucanases, one or more beta-glucosidases, and/or one or more cellobiohydrolases. The one or more other hemicellulases may be selected from, for example, one or more other beta-mannanases, one or more Alpha-L-arabinofuranosidases, one or more xylanases, and/or one or more beta-xylosidases. Accordingly the SgaMan1 polypeptide can be combined with one or more cellulases and/or one or more other hemicellulases to form an enzyme mixture/composition, which is a “physical mixture” or “admixture” of SgaMan1 and other polypeptides. The improved capacity observable or achievable with the co-expressed enzyme mixture is also observable or achievable with the admixture comprising SgaMan1.

As demonstrated herein, SgaMan1 polypeptides and compositions comprising SgaMan1 polypeptides have improved efficacy at conditions under which saccharification and degradation of lignocellulosic biomass take place. The improved efficacy of an enzyme composition comprising a SgaMan1 polypeptide is shown when its performance of hydrolyzing a given biomass substrate is compared to that of an otherwise comparable enzyme composition comprising certain other microbial beta-mannanases having similar pH optimums and/or temperature optimums. In certain embodiments, SgaMan1 polypeptides of the compositions and methods herein have at least about 5% (for example, at least about 5%, at least about 7%, at least about 10%, at least about 12%, at least about 13%, at least about 14%, at least about 15%, or more) increased capacity to hydrolyze a given lignocellulosic biomass substrate, which has optionally been subject to pretreatment, as compared to a benchmark GH5 beta-mannanase polypeptide XcaMan1 from Xanthomonas campestris comprising the amino acid sequence of SEQ ID NO: 4, or another GH5 SspMan2 polypeptide from Streptomyces sp., comprising the amino acid sequence of SEQ ID NO:5.

The performance of hydrolyzing a given biomass substrate can be measured by the extent or degree of liquefaction or viscosity reduction of the biomass substrate or the speed of such liquefaction or viscosity reduction of a given substrate having a particular solids level. The viscosity reduction and/or liquefaction and the rate thereof can be assessed using a method described in Example 10 (herein). As such a SgaMan1 polypeptide of the compositions and methods herein, when included in a given enzyme composition in a certain amount, confers at least a 5% higher viscosity reduction or level of liquefaction as compared to an otherwise same enzyme composition comprising the same amount of XcaMan1 or the same amount of SspMan2, under the same hydrolysis conditions and after the hydrolysis reaction is carried on for the same time period.

Aspects of the present compositions and methods include a recombinant polypeptide comprising an amino acid sequence that is at least 55% identical to the amino acid sequence of SEQ ID NO: 2, wherein the polypeptide has beta-mannanase activity. In some aspects, a SgaMan1 polypeptide and/or as it is applied in an enzyme composition or in a method to hydrolyze a lignocellulosic biomass substrate is (a) derived from, obtainable from, or produced by Streptococcus gallolyticus, for example, an endophytic bacteria Streptococcus sp.; (b) a recombinant polypeptide comprising an amino acid sequence that is at least 55% (e.g., at least 55%, 60%, 65%, 70%, 75%, 80%, 85%, 90%, 91%, 92%, 93%, 94%, 95%, 96%, 97%, 98%, 99%, or 100%) identical to the amino acid sequence of SEQ ID NO:2; (c) a recombinant polypeptide comprising an amino acid sequence that is at least 55% (e.g., at least 55%, 60%, 65%, 70%, 75%, 80%, 85%, 90%, 91%, 92%, 93%, 94%, 95%, 96%, 97%, 98%, 99%, or 100%) identical to the catalytic domain of SEQ ID NO:2, namely amino acid residues 22 to 487; (d) a recombinant polypeptide comprising an amino acid sequence that is at least 55% (e.g., at least 55%, 60%, 65%, 70%, 75%, 80%, 85%, 90%, 91%, 92%, 93%, 94%, 95%, 96%, 97%, 98%, 99%, or 100%) identical to the mature form of amino acid sequence of SEQ ID NO:3, namely amino acid residues 22 to 487 of SEQ ID NO:2; or (e) a fragment of (a), (b), (c) or (d) having beta-mannanase activity. In certain embodiments, it is provided a variant polypeptide having beta-mannanase activity, which comprises a substitution, a deletion and/or an insertion of one or more amino acid residues of SEQ ID NO:2 or SEQ ID NO:3. In certain embodiments, the polypeptide comprises an amino acid sequence that is at least 80% identical to the amino acid sequence of SEQ ID NO:2 or SEQ ID NO: 3. In certain embodiments, the polypeptide comprises an amino acid sequence that is at least 90% identical to the amino acid sequence of SEQ ID NO:2 or SEQ ID NO: 3. In certain embodiments, the polypeptide comprises an amino acid sequence that is at least 95% identical to the amino acid sequence of SEQ ID NO:2 or SEQ ID NO: 3. In certain embodiments, the polypeptide comprises an amino acid sequence that is at least 99% identical to the amino acid sequence of SEQ ID NO:2 or SEQ ID NO: 3.

In certain embodiments, the SgaMan1 polypeptide has a pH optimum of about pH 6.3. The SgaMan1 polypeptide retains greater than 70% of its maximum activity between pH 5.0 and pH 8.0.

In certain embodiments, the SgaMan1 polypeptide has an optimum temperature of about 58° C. The SgaMan1 polypeptide retains greater than 70% of its maximum activity between the temperatures of about 46° C. and about 60° C.

In certain embodiments, the SgaMan1 polypeptide has good thermostability. For example, when incubated at 55° C. for 2 hours, there was no loss of activity observed with SgaMan1. Furthermore, the SgaMan1 polypeptide retains about 50% of the beta-mannanase activity when incubated for about 2 hours at a temperature of about 57° C.

Aspects of the present compositions and methods include a composition comprising the recombinant SgaMan1 polypeptide as described herein and one or more cellulases. In some embodiments, the one or more cellulases may be selected from one or more endoglucanases, one or more cellobiohydrolases and/or one or more beta-glucosidases.

Aspects of the present compositions and methods include a composition comprising the recombinant SgaMan1 polypeptide as described herein and one or more hemicellulases. In some embodiments, the one or more other hemicellulases may be selected from one or more xylanases, beta-xylosidases, alpha-L-arabinofuranosidases and one or more other mannanases.

Aspects of the present compositions and methods include a composition comprising the recombinant SgaMan1 polypeptide as described herein and one or more cellulases and one or more other hemicellulases. For example, the one or more cellulases may be selected from endoglucanases, cellobiohydrolases, and/or beta-glucosidases, and the one or more other hemicellulases may include xylanases, beta-xylosidases, alpha-L-arabinofuranosidases and other mannanases.

As demonstrated herein, the SgaMan1 polypeptides described herein can impart, to an enzyme mixture or composition comprising a SgaMan1 polypeptide in addition to one or more cellulases, an improved capacity to hydrolyze, liquefy, saccharify, or degrade a given lignocellulosic biomass substrate, which has optionally been subject to pretreatment, and further optionally having had at least some of its xylan-containing components removed or separated from the glucan-containing components. Such improved capacity to hydrolyze, liquefy, saccharify, or degrade a given lignocellulosic biomass substrate may be evidenced by a measurably higher % glucan conversion, or reduced viscosity, achieved using a given enzyme composition comprising at least one cellulase, and a SgaMan1 polypeptide in an amount of as high as about 20 wt. % (for example, up to about 2 wt. %, up to about 5 wt. %, up to about 7 wt. %, up to about 10 wt. %, up to about 12 wt. %, up to about 15 wt. %, up to about 16 wt. %, up to about 17 wt. %, up to about 18 wt. %, up to about 19 wt. %, up to about 20 wt. %) of the enzyme composition, to hydrolyze a particular lignocellulosic biomass substrate, as compared to a counterpart enzyme composition comprising all the same other enzymes in the same proportion but comprising no SgaMan1 polypeptide.

The SgaMan1 polypeptides described herein can alternatively or additionally impart, to an enzyme mixture or composition comprising a SgaMan1 polypeptide in addition to one or more other hemicellulases, an improved capacity to hydrolyze, liquefy, saccharify, or degrade a given xylan-containing lignocellulosic biomass substrate, which has optionally been subject to pretreatment, and further optionally having at least had some of its xylan-containing components removed or separated from its glucan-containing components. Such improved capacity to hydrolyze, liquefy, saccharify, or degrade a given lignocellulosic biomass substrate may be evidenced by a measurably higher % xylan conversion achieved using a given enzyme composition comprising at least one other hemicellulase, and a SgaMan1 polypeptide in an amount of as high as about 20 wt. % (for example, up to about 2 wt. %, up to about 5 wt. %, up to about 7 wt. %, up to about 10 wt. %, up to about 12 wt. %, up to about 15 wt. %, up to about 16 wt. %, up to about 17 wt. %, up to about 18 wt. %, up to about 19 wt. %, up to about 20 wt. %) of the enzyme composition to hydrolyze a xylan-containing lignocellulosic biomass substrate or a xylan-containing component derived therefrom, as compared a counterpart enzyme composition comprising all the same other enzymes in the same proportion but comprising no SgaMan1 polypeptide.

Aspects of the present compositions and methods include a composition comprising a recombinant SgaMan1 polypeptide as detailed herein and a lignocellulosic biomass. Suitable lignocellulosic biomass may be, for example, derived from an agricultural crop, a byproduct of a food or feed production, a lignocellulosic waste product, a plant residue, including, for example, a grass residue, or a waste paper or waste paper product. Certain particularly suitable biomass may be one that comprises at least a measurable level of galactoglucomannan (GGM) and/or glucomannan (GM). Suitably the biomass may preferably be one that is rich in galactoglucomannan (GGM) and/or in glucomannan (GM), for example one that comprises at least about 0.5 wt. % (e.g., 0.5 wt. %, at least about 0.7 wt. %, at least about 1.0 wt. %, at least about 1.2 wt. %, at least about 1.5 wt. %, at least about 2.0 wt. %, at least about 2.5 wt. %, or more) GGM, or at least about 0.5 wt. % (e.g., 0.5 wt. %, at least about 0.7 wt. %, at least about 1.0 wt. %, at least about 1.2 wt. %, at least about 1.5 wt. %, at least about 2.0 wt. %, at least about 2.5 wt. %, or more) GM, or at least about 0.5 wt. % (e.g., 0.5 wt. %, at least about 0.7 wt. %, at least about 1.0 wt. %, at least about 1.2 wt. %, at least about 1.5 wt. %, at least about 2.0 wt. %, at least about 2.5 wt. %, at least about 3.0 wt. %, at least about 3.5 wt. %, at least about 4.0 wt. %, at least about 4.5 wt. %, at least about 5.0 wt. %, or more) of GGM and GM combined. In certain embodiments, the lignocellulosic biomass has been subject to one or more pretreatment steps in order to render xylan, hemicelluloses, cellulose and/or lignin material more accessible or susceptible to enzymes and thus more amendable to enzymatic hydrolysis. A suitable pretreatment method may be, for example, subjecting biomass material to a catalyst comprising a dilute solution of a strong acid and a metal salt in a reactor. See, e.g., U.S. Pat. Nos. 6,660,506, 6,423,145. Alternatively, a suitable pretreatment may be, for example, a multi-stepped process as described in U.S. Pat. No. 5,536,325. In certain embodiments, the biomass material may be subject to one or more stages of dilute acid hydrolysis using about 0.4% to about 2% of a strong acid, in accordance with the disclosures of U.S. Pat. No. 6,409,841. Further embodiments of pretreatment methods may include those described in, for example, U.S. Pat. No. 5,705,369; in Gould, (1984) Biotech. & Bioengr., 26:46-52; in Teixeira et al., (1999) Appl. Biochem & Biotech., 77-79:19-34; in International Published Patent Application WO2004/081185; or in U.S. Patent Publication No. 20070031918, or International Published Patent Application WO06110901. A non-limiting example of a suitable lignocellulosic biomass substrate is a softwood substrated pretreated using the US Department of Agriculture's SPORL protocol, as described in Example 10 herein. Another non-limiting example of a suitable lignocellulosic biomass substrate is an akaline KRAFT-pretreated softwood pulp FPP-27.

The present invention also pertains to isolated polynucleotides encoding polypeptides having beta-mannanase activity, wherein the isolated polynucleotides are selected from:
(1) a polynucleotide encoding a polypeptide comprising an amino acid sequence having at least 55% (e.g., at least 55%, 60%, 65%, 70%, 75%, 80%, 85%, 90%, 91%, 92%, 93%, 94%, 95%, 96%, 97%, 98%, 99%, or 100%) identity to SEQ ID NO:2 or to SEQ ID NO:3;
(2) a polynucleotide having at least 55% (e.g., at least 55%, 60%, 65%, 70%, 75%, 80%, 85%, 90%, 91%, 92%, 93%, 94%, 95%, 96%, 97%, 98%, 99%, or 100%) identity to SEQ ID NO:1, or hybridizes under medium stringency conditions, high stringency conditions, or very high stringency conditions to SEQ ID NO:1, or to a complementary sequence thereof

Aspects of the present compositions and methods include methods of making or producing a SgaMan1 polypeptide having beta-mannanase activity, employing an isolated nucleic acid sequence encoding the recombinant polypeptide comprising an amino acid sequence that is at least 55% identical (e.g., at least 55%, 60%, 65%, 70%, 75%, 80%, 85%, 90%, 91%, 92%, 93%, 94%, 95%, 96%, 97%, 98%, 99%, or 100%) to that of SEQ ID NO:2, or that of the mature sequence SEQ ID NO:3. In some embodiments, the polypeptide further comprises a native or non-native signal peptide such that the SgaMan1 polypeptide that is produced is secreted by a host organism, for example, the signal peptide comprises a sequence that is at least 90% identical to any one of SEQ ID NOs:9-37 to allow for heterologous expression in a variety of fungal host cells, yeast host cells and bacterial host cells. In certain embodiments the isolated nucleic acid comprises a sequence that is at least 55% (e.g., at least 55%, 60%, 65%, 70%, 75%, 80%, 85%, 90%, 91%, 92%, 93%, 94%, 95%, 96%, 97%, 98%, 99%, or 100%) identical to SEQ ID NO:1. In certain embodiments, the isolated nucleic acid further comprises a nucleic acid sequence encoding a signal peptide sequence. In certain embodiments, the signal peptide sequence may be one selected from SEQ ID NOs:9-37. In certain particular embodiments, a nucleic acid sequence encoding the signal peptide sequence of SEQ ID NO:13 or 14 is used to express a SgaMan1 polypeptide in Trichoderma reesei.

Aspects of the present compositions and methods include an expression vector comprising the isolated nucleic acid as described above in operable combination with a regulatory sequence.

Aspects of the present compositions and methods include a host cell comprising the expression vector. In certain embodiments, the host cell is a bacterial cell or a fungal cell.

Aspects of the present compositions and methods include a composition comprising the host cell described above and a culture medium. Aspects of the present compositions and methods include a method of producing a SgaMan1 polypeptide comprising: culturing the host cell described above in a culture medium, under suitable conditions to produce the beta-mannanase.

Aspects of the present compositions and methods include a composition comprising a SgaMan1 polypeptide in the supernatant of a culture medium produced in accordance with the methods for producing the beta-mannanase as described above.

In some aspects the present invention is related to nucleic acid constructs, recombinant expression vectors, engineered host cells comprising a polynucleotide encoding a polypeptide having beta-mannanase activity, as described above and herein. In further aspects, the present invention pertains to methods of preparing or producing the beta-mannanase polypeptides of the invention or compositions comprising such beta-mannanase polypeptides using the nucleic acid constructs, recombinant expression vectors, and/or engineered host cells. In particular, the present invention is related, for example, to a nucleic acid constructs comprising a suitable signal peptide operably linked to the mature sequence of the beta-mannanase that is at least 55% identical to SEQ ID NO:2 or to the mature sequence of SEQ ID NO:3, or is encoded by a polynucleotide that is at least 55% identical to SEQ ID NO:1, an isolated polynucleotide, a nucleic acid construct, a recombinant expression vector, or an engineered host cell comprising such a nucleic acid construct. In some embodiments, the signal peptide and beta-mannanase sequences are derived from different microorganisms.

Also provided is an expression vector comprising the isolated nucleic acid in operable combination with a regulatory sequence. Additionally, a host cell is provided comprising the expression vector. In still further embodiments, a composition is provided, which comprises the host cell and a culture medium.

In some embodiments, the host cell is a bacterial cell or a fungal cell.

In further embodiments, the SgaMan1 polypeptide is heterologously expressed by a host cell. For example, the SgaMan1 polypeptide is expressed by an engineered microorganism that is not Streptococcus gallolyticus. In some embodiments, the SgaMan1 polypeptide is co-expressed with one or more cellulase genes. In some embodiments, the SgaMan1 polypeptide is co-expressed with one or more other hemicellulase genes.

In some aspects, compositions comprising the recombinant SgaMan1 polypeptides of the preceding paragraphs and methods of preparing such compositions are provided. In some embodiments, the composition further comprises one or more cellulases, whereby the one or more cellulases are co-expressed by a host cell with the SgaMan1 polypeptide. In other embodiments, compositions comprising the SgaMan1 polypeptides may be an admixture of an isolated SgaMan1 polypeptide, optionally purified, physically blended with one or more cellulases and/or other enzymes. For example, the one or more cellulases can be selected from no or one or more beta-glucosidases, one or more cellobiohydrolyases, and/or one or more endoglucanases. In certain specific embodiments, such beta-glucosidases, cellobiohydrolases and/or endoglucanases, if present, can be co-expressed with the SgaMan1 polypeptide by a single host cell. In some embodiments, at least two of the two or more cellulases may be heterologous to each other or derived from different organisms. For example, the composition may comprise at least one beta-glucosidase and at least one cellobiohydrolase, whereby that beta-glucosidase and that cellobiohydrolase are not from the same microorganism. In some embodiments, one or more of the cellulases are endogenous to the host cell, but are overexpressed or expressed at a level that is different from that would otherwise be naturally-occurring in the host cell. For example, one or more of the cellulases may be a Trichoderma reesei CBH1 and/or CBH2, which are native to a Trichoderma reesei host cell, but either or both CBH1 and CBH2 are overexpressed or underexpressed when they are co-expressed in the Trichoderma reesei host cell with a SgaMan1 polypeptide.

In certain embodiments, the composition comprising the recombinant SgaMan1 polypeptide may further comprise one or more other hemicellulases, whereby the one or more other hemicellulases are co-expressed by a host cell with the SgaMan1 polypeptide. For example, the one or more other hemicellulases can be selected from one or more other beta-mannanases, one or more xylanases, one or more beta-xylosidases, and/or one or more L-arabinofuranosidases. In certain embodiments, such other mannanases, xylanases, beta-xylosidases and L-arabinofuranosidases, if present, can be co-expressed with the SgaMan1 polypeptide by a single host cell; or alternatively, one or more or all of such other mannanases, xylanases, beta-xylosidases and L-arabinofuranosidases, if present, are not co-expressed with the SgaMan1 polypeptides in a single host cell, but are rather physically mixed or blended together to form an enzyme composition after the individual enzymes are produced by their respective host cells.

In further aspects, the composition comprising the recombinant SgaMan1 polypeptide may further comprise one or more celluases and one or more other hemicelluases, whereby the one or more cellulases and/or one or more other hemicellulases are co-expressed by a host cell with the SgaMan1 polypeptide. For example, a SgaMan1 polypeptide may be co-expressed with one or more beta-glucosidases, one or more cellobiohydrolases, one or more endoglucanases, one or more endo-xylanases, one or more beta-xylosidases, and/or one or more L-arabinofuranosidases, in addition to other non-cellulase non-hemicellulase enzymes or proteins in the same host cell. Alternatively, the composition comprising the recombinant SgaMan1 polypeptide comprising one or more cellulases and one or more other hemicelulases may be prepared by physically mixing the SgaMan1 polypeptide with one or more cellulases and one or more other hemicellulases post production, whereby the SgaMan1 polypeptide and the one or more cellulases and one or more other hemicellulases are produced from different host cells. Aspects of the present compositions and methods thus include a composition comprising the host cell described above co-expressing a number of enzymes in addition to the SgaMan1 polypeptide and a culture medium. Alternatively, aspects of the present compositions and methods include a first composition comprising a first host cell expressing a SgaMan1, optionally in addition to one or more other enzymes/proteins, and a second composition comprising a second host cell expressing, for example, one or more cellulases and/or one or more other hemicellulases, and optionally a third composition comprising a third host cell expressing, for example, one or more other cellulases and/or one or more other hemicellulases that are different from those that are expressed by the first and second host cells. Such first, second, and third compositions resulting from enzyme production from the host cells, if appropriate, can suitably be physically blended or mixed to form an admixture of enzymes that form the present composition. Also provided are compositions that comprise the SgaMan1 polypeptide and the other enzymes produced in accordance with the methods herein in supernatant of a culture medium or culture media, as appropriate. Such supernatant of the culture medium can be used as is, with minimum or no post-production processing, which may typically include filtration to remove cell debris, cell-kill procedures, and/or ultrafiltration or other steps to enrich or concentrate the enzymes therein. Such supernatants are called “whole broths” or “whole cellulase broths” herein.

In further aspects, the present invention pertains to a method of applying or using the composition as described above under conditions suitable for degrading or converting a cellulosic material and for producing a substance from a cellulosic material.

In a further aspect, methods for degrading or converting a cellulosic material into fermentable sugars are provided, comprising: contacting the cellulosic material, preferably having already been subject to one or more pretreatment steps, with the SgaMan1 polypeptides or the compositions comprising such polypeptides of one of the preceding paragraphs to yield fermentable sugars.

Accordingly the instant specification is drawn to the following particular aspects:

In a first aspect, a recombinant polypeptide comprising an amino acid sequence that is at least 55% identical to the amino acid sequence of SEQ ID NO:2 or SEQ ID NO:3, wherein the polypeptide has beta-mannanase activity.

In a second aspect, the recombinant polypeptide of the first aspect, wherein the polypeptide improves the hydrolysis performance of a cellulase composition when the polypeptide constitutes up to 20 wt. % of the cellulase composition, wherein the improved hydrolysis performance comprises an at least about 5% faster viscosity reduction of a given lignocellulosic biomass substrate under the same hydrolysis conditions.

In a third aspect, the recombinant polypeptide of the first or the second aspect, wherein the polypeptide confers an increased viscosity reduction benefit to a cellulolytic hydrolysis enzyme composition comprising the polypeptide as compared to another similar cellulolytic hydrolysis enzyme composition comprising the same enzymes but a XcaMan1 comprising SEQ ID NO:4 in the place of the polypeptide.

In a fourth aspect, the recombinant polypeptide of the first or the second aspect, wherein the polypeptide confers an increased viscosity reduction benefit to a cellulolytic hydrolysis enzyme composition comprising the polypeptide as compared to another similar cellulolytic hydrolysis enzyme composition comprising the same enzymes but a SspMan2 comprising SEQ ID NO:5 in the place of the polypeptide.

In a fifth aspect, the recombinant polypeptide of any one of the first to the fourth aspects, wherein the polypeptide retains greater than 70% of the beta-mannanase activity when incubated at a pH range from pH 5.0 to pH 8.0.

In a sixth aspect, the recombinant polypeptide of any one of the first to fifth aspects, wherein the polypeptide has optimum beta-mannanase activity at a pH of about 6.3.

In a seventh aspect, the recombinant polypeptide of any one of the first to sixth aspects, wherein the polypeptide retains at least 70% or more of the beta-mannanase activity when incubated at a temperature of between 46° C. and 60° C.

In an eighth aspect, the recombinant polypeptide of any one of the first to seventh aspects, wherein the polypeptide has optimum beta-mannanase activity at a temperature of about 58° C. or above.

In a ninth aspect, the recombinant polypeptide of any one of the first to eighth aspects, wherein the polypeptide retains at least 50% of the beta-mannanase activity when incubated for about 2 hours at a temperature of about 57° C.

In a 10^{th} aspect, the recombinant polypeptide of any one of the first to ninth aspects, wherein the polypeptide comprises an amino acid sequence that is at least 60% identical to the amino acid sequence of SEQ ID NO:2 or SEQ ID NO:3.

In an 11^{th} aspect, the recombinant polypeptide of any one of the first to 10^{th} aspects, wherein the polypeptide comprises an amino acid sequence that is at least 65% identical to the amino acid sequence of SEQ ID NO:2 or SEQ ID NO:3.

In a 12^{th} aspect, the recombinant polypeptide of any one of the first to 11^{th} aspects, wherein the polypeptide comprises an amino acid sequence that is at least 70% identical to the amino acid sequence of SEQ ID NO:2 or SEQ ID NO:3.

In a 13^{th} aspect, an enzyme composition comprising the recombinant polypeptide of any one of the first to 12^{th} aspects, further comprising one or more cellulases.

In a 14^{th} aspect, the enzyme composition of the 13^{th} aspect, wherein the one or more cellulases are selected from one or more beta-glucosidases, one or more cellobiohydrolases, and one or more endoglucanases.

In a 15^{th} aspect, an enzyme composition comprising the recombinant polypeptide of any one of the first to 12^{th} aspects, further comprising one or more other hemicellulases.

In a 16^{th} aspect, the enzyme composition of the 15^{th} aspect, wherein the one or more other hemicellulases are selected from one or more other beta-mannanases, one or more one or more xylanases, one or more beta-xylosidases, and one or more L-arabinofuranosidases.

In a 17^{th} aspect, a nucleic acid encoding the recombinant polypeptide of any one of the first to 12^{th} aspects.

In an 18^{th} aspect, the nucleic acid of the 17^{th} aspect, wherein the polypeptide further comprises a signal peptide sequence.

In a 19^{th} aspect, the nucleic acid of the 18^{th} aspect, wherein the signal peptide sequence is selected from any one of SEQ ID NOs:9-37.

In a 20^{th} aspect, an expression vector comprising the nucleic acid of any one of the 17^{th} to 19^{th} aspects, in operable combination with a regulatory sequence.

In a 21^{st} aspect, a host cell comprising the expression vector of the 20^{th} aspect.

In a 22^{nd} aspect, the host cell of the 21^{st} aspect, wherein the host cell is a bacterial cell or a fungal cell.

In a 23^{rd} aspect, a composition comprising the host cell of the 21^{st} or 22^{nd} aspect and a culture medium.

In a 24^{th} aspect, a method of producing a beta-mannanase, comprising: culturing the host cell of the 21^{st} or 22^{nd} aspect, in a culture medium, under suitable conditions to produce the beta-mannanase.

In a 25^{th} aspect, a composition comprising the beta-mannanase produced in accordance with the method of the 24^{th} aspect in supernatant of the culture medium.

In a 26^{th} aspect, a method for hydrolyzing a lignocellulosic biomass substrate, comprising: contacting the lignocellulosic biomass substrate with the polypeptide of any one of the first to 12^{nd} aspects, or the composition of any one of the 13^{th} to 16^{th} and 25^{th} aspects, to yield glucose and other sugars.

In a 27^{th} aspect, the method of the 26^{th} aspect, wherein the lignocellulosic biomass substrate comprises up to about 20 wt. %, up to about 15%, or up to about 10 wt. % of galactoglucomannan and/or glucomannan.

In a 28^{th} aspect, a composition comprising the recombinant polypeptide of any one of the first to 12^{nd} aspects, and a lignocellulosic biomass substrate.

In a 29^{th} aspect, the composition of the 28^{th} aspect, wherein the lignocellulosic biomass substrate comprises up to about 20 wt. %, or up to about 15 wt. %, or up to about 10 wt. % of galactoglucomannan and/or glucomannan.

### BRIEF DESCRIPTION OF THE DRAWINGS

FIG. 1 depicts a map of the p2JM103BBI vector.

FIG. 2 depicts a map of the p2JM(aprE-SgaMan1) construct.

FIG. 3 depicts a pH profile of SgaMan1. The effect of pH on beta-mannanase activity of SgaMan1 was measured at 50° C. for 10 minutes using 1% locust bean gum as 2 to 9 at 50° C. for 10 min with locust bean gum as the substrate 2 to 9 at 50° C. for 10 min with locust bean gum as the substrate substrate in 50 mM sodium citrate and 50 mM sodium phosphate buffer adjusted to individual pH values ranging between pH 2-9. The mannanase activity of the SgaMan1 polypeptide at its pH optimum was normalized to 100%, and the mannanase activity of the same polypeptide at other pH values were depicted as relative activity to that at the pH optimum.

FIG. 4 depicts a temperature profile of SgaMan1. The effect of temperature change on beta-mannanase activity of SgaMan1 was measured at individual temperature values ranging between 40° C. and 90° C. for 10 minutes using 1% locust bean gum as substrate in a 50 mM sodium citrate buffer, at pH 6.0. The mannanase activity of the SgaMan1 polypeptide at its temperature optimum was normalized to 100%, and the mannanase activity of the same polypeptide at other temperature values were depicted as relative activity to that at the temperature optimum.

FIG. 5 depicts a thermostability profile of SgaMan1. The thermostability of SgaMan1 was determined by incubation in 50 mM sodium citrate buffer at pH 6.0 at a set temperature within the range of 40° C. and 90° C. for 2 hours. After incubation, the remaining mannanase activity at each of the incubation temperature was measured. The activity measured from a control sample of the SgaMan1 polypeptide kept on ice for the same 2 hours was used as the 100% activity to normalize the residual activity measurements.

FIG. 6 depict the comparison of levels of hydrolysis and viscosity reduction achieved by a commercial cellulase/hemicellulase composition Accellerase® TRIO˜ vs. a blend of 9 parts Accellerase® TRIO™ with 1 part (i.e., 10 wt. %) of a SgaMan1 polypeptide, as compared to the same blend of Accellerase® TRIO™ with each of two other beta-mannanases of GHS, a Xanthomonas campestris beta-mannanase of SEQ ID NO:4 (“XcaMan1”) and a Streptomyces sp. beta-mannanase of SEQ ID NO:5 (“SspMan2”), of a given biomass substrate, namely the alkaline KRAFT-pretreated softwood substrate FPP-27, under the same hydrolysis conditions and at different durations of reaction. Details of the experiments are found in Example 9.

FIG. 7 describes sequences referenced elsewhere herein.

### DETAILED DESCRIPTION

Described herein are compositions and methods relating to a recombinant beta-mannanase belonging to glycosyl hydrolase family 5 from Streptococcus gallolyticus. The present compositions and methods are based, in part, on the observations that recombinant SgaMan1 polypeptides confer to a cellulase and/or hemicellulase composition comprising at least one cellulase and/or at least one other hemicellulase, an improved capacity to hydrolyze a lignocellulosic biomass material or feedstock than other known beta-mannanases of similar pH optimums and/or temperature optimums. The present compositions and methods are also based on the observation that recombinant SgaMan1 polypeptides confers rapid viscosity reduction when compositions comprising the polypeptides are used to hydrolyze suitable lignocellulosic biomass substrates, especially when such substrates are treated at high solids levels, and when such substrates contain measurable level of galactoglucomannan (GGM) and/or glucomannan (GM). Adequate liquefaction and viscosity reduction is necessary to facilitate mass transfer limitations of hydrolysis. Viscosity reduction of the hydrolysate can enable greater substrate/enzyme interactions resulting in improved hydrolysis rates. Highly viscous systems can significantly decrease the hydrolytic efficiencies of the enzymes. As such, the capacity of SgaMan1 to confer viscosity reduction benefits to a cellulolytic enzyme composition makes such polypeptides or variants thereof, suitable for use in numerous processes, including, for example, in the conversion or hydrolysis of a lignocellulosic biomass feedstock.

Before the present compositions and methods are described in greater detail, it is to be understood that the present compositions and methods are not limited to particular embodiments described, as such may, of course, vary. It is also to be understood that the terminology used herein is for the purpose of describing particular embodiments only, and is not intended to be limiting, since the scope of the present compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present compositions and methods are not entitled to antedate such publication by virtue of prior invention. Further, the dates of publication provided may be different from the actual publication dates which may need to be independently confirmed.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “recombinant,” when used in reference to a subject cell, nucleic acid, polypeptides/enzymes or vector, indicates that the subject has been modified from its native state. Thus, for example, recombinant cells express genes that are not found within the native (non-recombinant) form of the cell, or express native genes at different levels or under different conditions than found in nature. Recombinant nucleic acids may differ from a native sequence by one or more nucleotides and/or are operably linked to heterologous sequences, e.g., a heterologous promoter, signal sequences that allow secretion, etc., in an expression vector. Recombinant polypeptides/enzymes may differ from a native sequence by one or more amino acids and/or are fused with heterologous sequences. A vector comprising a nucleic acid encoding a beta-mannanase is, for example, a recombinant vector.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

“Beta-mannanase” means a polypeptide or polypeptide domain of an enzyme that has the ability to catalyze the cleavage or hydrolysis of (1→4)-beta-D-mannosidic linkages of mannans, galactomannans, and glucomannans.

As used herein, “SgaMan1 ” or “a SgaMan1 polypeptide” refers to a beta-mannanase belonging to glycosyl hydrolase family 5 (e.g., a recombinant beta-mannanase) derived from Streptococcus gallolyticus (and variants thereof), that confers surprising improvements to a cellulase and/or hemicellulase composition in the composition's capability to hydrolyze a lignocellulosic biomass substrate, optionally pretreated, when compared to other known beta-mannanases of similar pH optimums and/or temperature optimums. The SgaMan1 polypeptide can substitute a substantial portion, e.g., up to about 20 wt. % (e.g., up to about 20 wt. %, up to about 15 wt. %, up to about 10 wt. %, up to about 9 wt. %, up to about 8 wt. %, up to about 7 wt. %, up to about 6 wt. %, up to about 5 wt. %, up to about 4 wt. %, up to about 3 wt. %, up to about 2 wt. %, up to about 1 wt. %) of a cellulase and/or hemicellulase mixture and achieve equal or better hydrolysis of a given lignocellulosic biomass substrate under the same conditions. This allows the use of less cellulases/hemicellulases and more efficient biomass hydrolysis, thus making the overall cellulosic biomass conversion process more economically feasible and sustainable. The SgaMan1 polypeptide herein was also surprisingly found to confer rapid viscosity reduction or liquefaction, particularly prominently when the biomass substrate is treated with enzyme at high solids levels. According to aspects of the present compositions and methods, SgaMan1 polypeptides include those having the amino acid sequence depicted in SEQ ID NO:2, as well as derivative or variant polypeptides having at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:2, or to the mature sequence SEQ ID NO:2, or to a fragment of at least 80 residues in length of SEQ. ID NO:2, wherein the SgaMan1 polypeptides not only have beta-mannanase activity and capable of catalyzing the conversion hydrolysis of (1→4)-beta-D-mannosidic linkages of mannans, galactomannans, and glucomannans, but also have higher beta-mannanase activity than other beta-mannases of similar pH optimums and/or temperature optimums, and confer rapid viscosity reduction and liquefaction of high solids biomass substrates, a property that has not been observed with other known beta-mannanases.

“Family 5 glycosyl hydrolase” or “GH5” refers to polypeptides falling within the definition of glycosyl hydrolase family 5 according to the classification by Henrissat, Biochem. J. 280:309-316 (1991), and by Henrissat & Cairoch, Biochem. J., 316:695-696 (1996). Similarly, “Family 26 glycosyl hydrolase” or “GH26” refers to polypeptides falling within the definition of glycosyl hydrolase family 26 according to the classification by Henrissat, Biochem. J. 280:309-316 (1991), and by Henrissat & Cairoch, Biochem. J., 316:695-696 (1996).

SgaMan1 polypeptides according to the present compositions and methods described herein can be isolated or purified. By purification or isolation is meant that the SgaMan1 polypeptide is altered from its natural state by virtue of separating the SgaMan1 from some or all of the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to the SgaMan1 -containing composition which provide additional benefits, for example, activating agents, anti-inhibition agents, desirable ions, compounds to control pH or other enzymes or chemicals.

As used herein, “microorganism” refers to a bacterium, a fungus, a virus, a protozoan, and other microbes or microscopic organisms.

As used herein, a “derivative” or “variant” of a polypeptide means a polypeptide, which is derived from a precursor polypeptide (e.g., the native polypeptide) by addition of one or more amino acids to either or both the C- and N-terminal end, substitution of one or more amino acids at one or a number of different sites in the amino acid sequence, deletion of one or more amino acids at either or both ends of the polypeptide or at one or more sites in the amino acid sequence, or insertion of one or more amino acids at one or more sites in the amino acid sequence. The preparation of a SgaMan1 derivative or variant may be achieved in any convenient manner, e.g., by modifying a DNA sequence which encodes the native polypeptides, transformation of that DNA sequence into a suitable host, and expression of the modified DNA sequence to form the derivative/variant SgaMan1. Derivatives or variants further include SgaMan1 polypeptides that are chemically modified, e.g., glycosylation or otherwise changing a characteristic of the SgaMan1 polypeptide. While derivatives and variants of SgaMan1 are encompassed by the present compositions and methods, such derivates and variants will confer improved saccharification or liquefaction properties under the same lignocellulosic biomass substrate hydrolysis conditions, when compared to that of a number of other beta-mannanases having similar pH optimums and/or temperature optimums, for example the XcaMan1 having the sequence of SEQ ID NO:4, or the SspMan2, having the sequence of SEQ ID NO:5. In some embodiments, such derivatives and variants will confer rapid viscosity reduction and liquefaction to a cellulase and/or hemicellulase composition, capable of achieving, for example, at least 10% (e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 95%, at least 100%, or even more) improved viscosity reduction or higher liquefaction within the same time period after the biomass substrate is subject to an enzyme composition comprising a SgaMan1 polypeptide herein, as compared to when that same biomass substrate is subject to a counterpart enzyme composition having the same amounts, proportion, and types of enzymes except that the composition does not comprise the SgaMan1 polypeptide.

In certain aspects, a SgaMan1 polypeptide of the compositions and methods herein may also encompasses functional fragment of a polypeptide or a polypeptide fragment having beta-mannanase activity, which is derived from a parent polypeptide, which may be the full length polypeptide comprising or consisting of SEQ ID NO:2, or the mature sequence comprising or consisting SEQ ID NO:3. The functional polypeptide may have been truncated either in the N-terminal region, or the C-terminal region, or in both regions to generate a fragment of the parent polypeptide. For the purpose of the present disclosure, a functional fragment must have at least 20%, more preferably at least 30%, 40%, 50%, or preferably, at least 60%, 70%, 80%, or even more preferably at least 90% of the beta-mannanase activity of that of the parent polypeptide.

In certain aspects, a SgaMan1 derivative/variant will have anywhere from 55% to 99% (or more) amino acid sequence identity to the amino acid sequence of SEQ ID NO:2, or to the mature sequence SEQ ID NO:3, e.g., 55%, 60%, 65%, 70%, 75%, 80%, 85%, 86%, 87%, 88%, 89%, 90%, 91%, 92%, 93%, 94%, 95%, 96%, 97%, 98%, or 99% amino acid sequence identity to the amino acid sequence of SEQ. ID NO:2 or to the mature sequence SEQ ID NO:3. In some embodiments, amino acid substitutions are “conservative amino acid substitutions” using L-amino acids, wherein one amino acid is replaced by another biologically similar amino acid. Conservative amino acid substitutions are those that preserve the general charge, hydrophobicity/hydrophilicity, and/or steric bulk of the amino acid being substituted. Examples of conservative substitutions are those between the following groups: Gly/Ala, Val/Ile/Leu, Lys/Arg, Asn/Gln, Glu/Asp, Ser/Cys/Thr, and Phe/Trp/Tyr. A derivative may, for example, differ by as few as 1 to 10 amino acid residues, such as 6-10, as few as 5, as few as 4, 3, 2, or even 1 amino acid residue. In some embodiments, a SgaMan1 derivative may have an N-terminal and/or C-terminal deletion, where the SgaMan1 derivative excluding the deleted terminal portion(s) is identical to a contiguous sub-region in SEQ ID NO: 2 or SEQ ID NO:3.

As used herein, “percent (%) sequence identity” with respect to the amino acid or nucleotide sequences identified herein is defined as the percentage of amino acid residues or nucleotides in a candidate sequence that are identical with the amino acid residues or nucleotides in a SgaMan1 sequence, after aligning the sequences and introducing gaps, if necessary, to achieve the maximum percent sequence identity, and not considering any conservative substitutions as part of the sequence identity.

By “homologue” shall mean an entity having a specified degree of identity with the subject amino acid sequences and the subject nucleotide sequences. A homologous sequence is taken to include an amino acid sequence that is at least 50%, 55%, 60%, 65%, 70%, 75%, 80%, 85%, 86%, 87%, 88%, 89%, 90%, 91%, 92%, 93%, 94%, 95%, 96%, 97%, 98% or even 99% identical to the subject sequence, using conventional sequence alignment tools (e.g., Clustal, BLAST, and the like). Typically, homologues will include the same active site residues as the subject amino acid sequence, unless otherwise specified.

Methods for performing sequence alignment and determining sequence identity are known to the skilled artisan, may be performed without undue experimentation, and calculations of identity values may be obtained with definiteness. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Protein Sequence and Structure 5:Suppl. 3 (National Biomedical Research Foundation, Washington, D.C.). A number of algorithms are available for aligning sequences and determining sequence identity and include, for example, the homology alignment algorithm of Needleman et al. (1970) J Mol. Biol. 48:443; the local homology algorithm of Smith et al. (1981) Adv. Appl. Math. 2:482; the search for similarity method of Pearson et al. (1988) Proc. Natl. Acad. Sci. 85:2444; the Smith-Waterman algorithm (Meth. Mol. Biol. 70:173-187 (1997); and BLASTP, BLASTN, and BLASTX algorithms (see Altschul et al. (1990) J Mol. Biol. 2/5:403-410).

Computerized programs using these algorithms are also available, and include, but are not limited to: ALIGN or Megalign (DNASTAR) software, or WU-BLAST-2 (Altschul et al., (1996) Meth. Enzym., 266:460-480); or GAP, BESTFIT, BLAST, FASTA, and TFASTA, available in the Genetics Computing Group (GCG) package, Version 8, Madison, Wis., USA; and CLUSTAL in the PC/Gene program by Intelligenetics, Mountain View, Calif. Those skilled in the art can determine appropriate parameters for measuring alignment, including algorithms needed to achieve maximal alignment over the length of the sequences being compared. Preferably, the sequence identity is determined using the default parameters determined by the program. Specifically, sequence identity can determined by using Clustal W (Thompson J. D. et al. (1994) Nucleic Acids Res. 22:4673-4680) with default parameters, i.e.:

Gap opening penalty: 10.0

Gap extension penalty: 0.05

Protein weight matrix: BLOSUM series

DNA weight matrix: IUB

Delay divergent sequences %: 40

Gap separation distance: 8

DNA transitions weight: 0.50

List hydrophilic residues: GPSNDQEKR

Use negative matrix: OFF

Toggle Residue specific penalties: ON

Toggle hydrophilic penalties: ON

Toggle end gap separation penalty OFF

As used herein, “expression vector” means a DNA construct including a DNA sequence which is operably linked to a suitable control sequence capable of affecting the expression of the DNA in a suitable host. Such control sequences may include a promoter to affect transcription, an optional operator sequence to control transcription, a sequence encoding suitable ribosome-binding sites on the mRNA, and sequences which control termination of transcription and translation. Different cell types may be used with different expression vectors. An exemplary promoter for vectors used in Bacillus subtilis is the AprE promoter; an exemplary promoter used in Streptomyces lividans is the A4 promoter (from Aspergillus niger); an exemplary promoter used in E. coli is the Lac promoter, an exemplary promoter used in Saccharomyces cerevisiae is PGK1, an exemplary promoter used in Aspergillus niger is glaA, and an exemplary promoter for Trichoderma reesei is cbh1. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the present compositions and methods are intended to include other forms of expression vectors which serve equivalent functions and which are, or become, known in the art. Thus, a wide variety of host/expression vector combinations may be employed in expressing the DNA sequences described herein. Useful expression vectors, for example, may consist of segments of chromosomal, non-chromosomal and synthetic DNA sequences such as various known derivatives of SV40 and known bacterial plasmids, e.g., plasmids from E. coli including col E1, pCR1, pBR322, pMb9, pUC 19 and their derivatives, wider host range plasmids, e.g., RP4, phage DNAs e.g., the numerous derivatives of phage λ, e.g., NM989, and other DNA phages, e.g., M13 and filamentous single stranded DNA phages, yeast plasmids such as the 2μ plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors of the present compositions and methods are known in the art and are described generally in, for example, Sambrook et al., Molecular Cloning: A Laboratory Manual, Second Edition, Cold Spring Harbor Press (1989). Often, such expression vectors including the DNA sequences described herein are transformed into a unicellular host by direct insertion into the genome of a particular species through an integration event (see e.g., Bennett & Lasure, More Gene Manipulations in Fungi, Academic Press, San Diego, pp. 70-76 (1991) and articles cited therein describing targeted genomic insertion in fungal hosts).

As used herein, “host strain” or “host cell” means a suitable host for an expression vector including DNA according to the present compositions and methods. Host cells useful in the present compositions and methods are generally prokaryotic or eukaryotic hosts, including any transformable microorganism in which expression can be achieved. Specifically, host strains may be Bacillus subtilis, Bacillus licheniformis, Streptomyces lividans, Escherichia coli, Trichoderma reesei, Saccharomyces cerevisiae, Aspergillus niger, Aspergillus oryzae, Chrysosporium lucknowence, Myceliophthora thermophila, and various other microbial cells. Host cells are transformed or transfected with vectors constructed using recombinant DNA techniques. Such transformed host cells may be capable of one or both of replicating the vectors encoding SgaMan1 (and its derivatives or variants (mutants)) and expressing the desired peptide product. In certain embodiments according to the present compositions and methods, “host cell” means both the cells and protoplasts created from the cells of Trichoderma sp.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection,” “transformation,” or “transduction,” as known in the art.

A “host strain” or “host cell” is an organism into which an expression vector, phage, virus, or other DNA construct, including a polynucleotide encoding a polypeptide of interest (e.g., a beta-mannanase) has been introduced. Exemplary host strains are microbial cells (e.g., bacteria, filamentous fungi, and yeast) capable of expressing the polypeptide of interest. The term “host cell” includes protoplasts created from cells.

The term “heterologous” with reference to a polynucleotide or polypeptide refers to a polynucleotide or polypeptide that does not naturally occur in a host cell.

The term “endogenous” with reference to a polynucleotide or polypeptide refers to a polynucleotide or polypeptide that occurs naturally in the host cell.

The term “expression” refers to the process by which a polypeptide is produced based on a nucleic acid sequence. The process includes both transcription and translation.

As used herein, “signal sequence” means a sequence of amino acids bound to the N-terminal portion of a protein which facilitates the secretion of the mature form of the protein outside of the cell. This definition of a signal sequence is a functional one. The mature form of the extracellular protein lacks the signal sequence which is cleaved off during the secretion process. While the native signal sequence of SgaMan1 may be employed in aspects of the present compositions and methods, other non-native signal sequences may be employed (e.g., one selected from SEQ ID NOs:9-37).

The beta-mannanase polypeptides of the invention may be referred to as “precursor,” “immature,” or “full-length,” in which case they include a signal sequence, or may be referred to as “mature,” in which case they lack a signal sequence. Mature forms of the polypeptides are generally the most useful. Unless otherwise noted, the amino acid residue numbering used herein refers to the mature forms of the respective amylase polypeptides. The beta-mannanase polypeptides of the invention may also be truncated to remove the N or C-termini, so long as the resulting polypeptides retain beta-mannanase activity.

The beta-mannanase polypeptides of the invention may also be a “chimeric” or “hybrid” polypeptide, in that it includes at least a portion of a first beta-mannanase polypeptide, and at least a portion of a second beta-mannanase polypeptide (such chimeric beta-mannanase polypeptides may, for example, be derived from the first and second beta-mannanase using known technologies involving the swapping of domains on each of the beta-mannanase). The present beta-mannanase polypeptides may further include heterologous signal sequence, an epitope to allow tracking or purification, or the like. When the term of “heterologous” is used to refer to a signal sequence used to express a polypeptide of interest, it is meant that the signal sequence is, for example, derived from a different microorganism as the polypeptide of interest. Examples of suitable heterologous signal sequences for expressing the SgaMan1 polypeptides herein, may be, for example, those from Trichoderma reesei, other Trichoderma spp., Aspergillus niger, Aspergillus oryzae, other Aspergillus spp., Chrysosporium, and other organisms, those from Bacillus subtilis, Bacillus licheniformis, other Bacillus species, E.coli., or other suitable microbes.

As used herein, “functionally attached” or “operably linked” means that a regulatory region or functional domain having a known or desired activity, such as a promoter, terminator, signal sequence or enhancer region, is attached to or linked to a target (e.g., a gene or polypeptide) in such a manner as to allow the regulatory region or functional domain to control the expression, secretion or function of that target according to its known or desired activity.

As used herein, the terms “polypeptide” and “enzyme” are used interchangeably to refer to polymers of any length comprising amino acid residues linked by peptide bonds. The conventional one-letter or three-letter codes for amino acid residues are used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, “wild-type” and “native” genes, enzymes, or strains, are those found in nature.

The terms “wild-type,” “parental,” or “reference,” with respect to a polypeptide, refer to a naturally-occurring polypeptide that does not include a man-made substitution, insertion, or deletion at one or more amino acid positions. Similarly, the term “wild-type,” “parental,” or “reference,” with respect to a polynucleotide, refers to a naturally-occurring polynucleotide that does not include a man-made nucleoside change. However, a polynucleotide encoding a wild-type, parental, or reference polypeptide is not limited to a naturally-occurring polynucleotide, but rather encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues. They may be defined by their level of primary amino acid sequence homology/identity with a parent polypeptide. Suitably, variant polypeptides have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity to a parent polypeptide.

As used herein, a “variant polynucleotide” encodes a variant polypeptide, has a specified degree of homology/identity with a parent polynucleotide, or hybridized under stringent conditions to a parent polynucleotide or the complement thereof. Suitably, a variant polynucleotide has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity to a parent polynucleotide or to a complement of the parent polynucleotide. Methods for determining percent identity are known in the art and described above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm −5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Alternatively, or in addition, hybridization conditions can be based upon the salt or ionic strength conditions of hybridization, and/or upon one or more stringency washes, e.g.: 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used).

As used herein, the term “hybridization” refers to the process by which a strand of nucleic acid joins with a complementary strand through base pairing, as known in the art. More specifically, “hybridization” refers to the process by which one strand of nucleic acid forms a duplex with, i.e., base pairs with, a complementary strand, as occurs during blot hybridization techniques and PCR techniques. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while intermediate or low stringency hybridization can be used to identify or detect polynucleotide sequence homologs.

Intermediate and high stringency hybridization conditions are well known in the art. For example, intermediate stringency hybridizations may be carried out with an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denatured sheared salmon sperm DNA, followed by washing the filters in 1×SSC at about 37-50° C. High stringency hybridization conditions may be hybridization at 65° C. and 0.1×SSC (where 1×SSC=0.15 M NaCl, 0.015 M Na₃ citrate, pH 7.0). Alternatively, high stringency hybridization conditions can be carried out at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. And very high stringent hybridization conditions may be hybridization at 68° C. and 0.1×SSC. Those of skill in the art know how to adjust the temperature, ionic strength, etc. as necessary to accommodate factors such as probe length and the like.

A nucleic acid encoding a variant beta-mannase may have a Tₘ reduced by 1° C.-3° C. or more compared to a duplex formed between the nucleotide of SEQ ID NO:1 and its identical complement.

The phrase “substantially similar” or “substantially identical,” in the context of at least two nucleic acids or polypeptides, means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences may include a promoter to effect transcription, an optional operator sequence to control such transcription, a sequence encoding suitable mRNA ribosome binding sites and/or sequences that control termination of transcription and translation. The vector may be a plasmid, a phage particle, or a potential genomic insert. Once transformed into a suitable host, the vector may replicate and function independently of the host genome, or may, in some instances, integrate into the host genome.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in a manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a polypeptide, and which facilitates the secretion of the mature form of the polypeptide from the cell. The mature form of the extracellular polypeptide lacks the signal sequence which is cleaved off during the secretion process.

The term “selective marker” or “selectable marker,” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element,” refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally cells of prokaryotic or eukaryotic hosts that are transformed or transfected with vectors constructed using recombinant DNA techniques known in the art. Transformed host cells are capable of either replicating vectors encoding the polypeptide variants or expressing the desired polypeptide variant. In the case of vectors, which encode the pre- or pro-form of the polypeptide variant, such variants, when expressed, are typically secreted from the host cell into the host cell medium.

The term “introduced,” in the context of inserting a nucleic acid sequence into a cell, means transformation, transduction, or transfection. Means of transformation include protoplast transformation, calcium chloride precipitation, electroporation, naked DNA, and the like as known in the art. (See, Chang and Cohen (1979) Mol. Gen. Genet. 168:111-115; Smith et al., (1986) Appl. Env. Microbiol. 51:634; and the review article by Ferrari et al., in Harwood, Bacillus, Plenum Publishing Corporation, pp. 57-72, 1989).

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

Other technical and scientific terms have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, NY 1991).

The beta-mannanase enzyme SgaMan1 from Streptococcus gallolyticus UCN34 (SEQ ID NO:2) has the following amino acid sequence:

[symbol]

[symbol]

QGVKADEIVSGQTYKITAKHSGKALDV
ASKATYAGANVQQWSYNGGTNQQWKVVDTGDGYYKLVSVNSGKVLDVAS
AATYNGANVQQWDDTNGTCQRWKIVDTGDGSYKLISAVSGKALDVAANS
SDDGANVQQWDDNGGWNQKWEFTKVSDSGNTGGDTGGDTTTPVSTSGFY
TNGTTLYDAKGNPFVMRGINHAHSWYQGYEDTAIPAIAKTGANCVRLVI
TDGQQYSKTSLSELQKLIQLCKDNKLIAIVEVHDATGSDNIQDLEKAAD
YWIEMKSALIGNEANVILNIANEWGGAWDSSNWAAGYQQVIPKLRNAGI
KNTLMVDCAGWGQYPQSIAEAGQSVVQADSQHNIMFSIHMYEYAGAYGA
VQSNIDSALNVGVPLCIGEFGIRHTNGDVDYKTIMSYSQSKGVGYLGWS
WKGNGDTWAYLDIAQDWSGSQLTEQGNAIIYDQNGIKNTSKRCTIFD

The mature beta-mannanase enzyme, as based on the removal of the predicted signal peptide sequence of SEQ ID NO:3:

QGVKADEIVSGQTYKITAKHSGKALDVASKATYAGANVQQWSYNGGTNQ
QWKVVDTGCGYYKLVSVNSGKVLDVASAATYNGANVQQWDDTNGTCQRW
KIVDTGDGSYKLISAVSGKALDVAANSSDDGANVQQWDDNGGWNQKWEF
TKVSDSGNTGGDTGGDTTTPVSTSGFYTNGTTLYDAKFNPFVMRGINHA
HSWYQGYEDTAIPAIAKTGANCVRLVITDGQQYSKTSLSELQKLIQLCK
DNKLIAIVEVHDATGSDNIQDLEKAADYWIEMKSALIGNEANVILNIAN
EWGGAWDSSNWAAGYQQVIPKLRNAGIKTNTLMVDCAGWGQYPQSIAEA
GQSVVQADSQHNIMFSIHMYEYAGAYGAVQSNIDSALNVGVPLCIGEFG
IRHTNGDVDYKTIMSYSQSKGVGYLGWSWKGNGDTWAYLDIAQDWSGSQ
LTEQGNAIIYDQNGIKNTSKRCTIFD

A number of other bacterial beta-mannanases having similar pH optimums and/or temperature optimums have been used as benchmark molecules herein, including a beta-mannanase of Xanthomonas capestris, called “XcaMan1” herein, having the following amino acid sequence (SEQ ID NO: 4):

GLSVSGTQLKESNGNTLILRGINLPHAWFADRTDAALAQIAATGANSVR
VVLSSGHRWNRTPEAEVARIIARCKALGLIAVLEVHDTTGYGEDGAAGS
LANAASYWTSVRTALVGQEDYVIINIGNEPFGNQLSASEWVNGHANAIA
TLRGAGLTHALMVDAPNWGQDWQFYMRDNAAALLARDSRRNLIFSVHMY
EVFGSDAVVDSYLRTFRSNNLALVVGEFGADHRGAPVDEAAIMRRAREY
GVGYLGWSWSGNDSSTQSLDIVLGWDPARLSSWGRLSIQGPDGIAATSR
RARVFGARVRAME

Benchmark beta-mannanases also include a GH5 beta-mannanase SspMan2 from Streptomyces sp., having the following amino acid sequence (SEQ ID NO:5):

AEAATGIRVGNGRVYEANGNEFVMRGVNHAHAWYPNRTGSIAHIKAKGA
NTVRVVLANGDRWTRTSASEVSSIIGQCKQNRLICVLEVHDTTGYGEDG
AATSLSRAADYWIGVKSALEGQENYVVINIGNEPFGNNGYDRWTSDTIA
AVQKLRNAGFDHALMVDAPNWGQDWSNTMRNNASTVFNSDPDRNTIFSI
HMYGVYNTASEVQSYLNHFVGNRLPIVVGEFGHNHGDGDPDENAIMATA
QSLRVGYLGWSWSGNGGGVEYLDMVNGFDPNSLTGWGQRFFNGANGISA
TSREATVYGGGSGGGSGGTAPNGYPYCVDGSASDPDGDGWGWENQRSCV
VRGSAADG

### Beta-Mannanase Polypeptides, Polynucleotides, Vectors, and Host Cells

### SgaMan1 Polypeptides

In one aspect, the present compositions and methods provide a recombinant SgaMan1 beta-mannanase polypeptide, fragments thereof, or variants thereof having beta-mannanase activity. An example of a recombinant beta-mannanase polypeptide was isolated from Streptococcus gallolyticus. The mature SgaMan1 polypeptide has the amino acid sequence set forth as SEQ ID NO:3. Similar, substantially similar SgaMan1 polypeptides may occur in nature, e.g., in other strains or isolates of Streptococcus gallolyticus, or Streptococcus spp. These and other recombinant SgaMan1 polypeptides are encompassed by the present compositions and methods.

In some embodiments, the recombinant SgaMan1 polypeptide is a variant SgaMan1 polypeptide having a specified degree of amino acid sequence identity to the exemplified SgaMan1 polypeptide, e.g., at least 55%, 60%, 65%, 70%, 75%, 80%, 85%, 90%, 91%, 92%, 93%, 94%, 95%, 96%, 97%, 98%, or even at least 99% sequence identity to the amino acid sequence of SEQ ID NO:2 or to the mature sequence SEQ ID NO:3. Sequence identity can be determined by amino acid sequence alignment, e.g., using a program such as BLAST, ALIGN, or CLUSTAL, as described herein.

In certain embodiments, the recombinant SgaMan1 polypeptides are produced recombinantly, in a microorganism, for example, in a bacterial or fungal host organism, while in others the SgaMan1 polypeptides are produced synthetically, or are purified from a native source (e.g., Streptococcus gallolyticus).

In certain embodiments, the recombinant SgaMan1 polypeptide includes substitutions that do not substantially affect the structure and/or function of the polypeptide. Examples of these substitutions are conservative mutations, as summarized in Table I.

TABLE I
Amino Acid Substitutions

| Original | | |
|---|---|---|
| Residue | Code | Acceptable Substitutions |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, |
| | | D-Leu, Val, D-Val |
| Phenylalanine | F | D-Phe, Tyr, D-Thr, L-Dopa, His, D-His, |
| | | Trp, D-Trp, Trans-3,4, |
| | | or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a nucleic acid encoding a recombinant SgaMan1 polypeptide, and then expressing the variant polypeptide in an organism. Substitutions involving non-naturally occurring amino acids or chemical modifications to amino acids are generally made by chemically modifying a SgaMan1 polypeptide after it has been synthesized by an organism.

In some embodiments, variant recombinant SgaMan1 polypeptides are substantially identical to SEQ ID NO:2 or SEQ ID NO:3, meaning that they do not include amino acid substitutions, insertions, or deletions that do not significantly affect the structure, function, or expression of the polypeptide. Such variant recombinant SgaMan1 polypeptides will include those designed to circumvent the present description. In some embodiments, variants recombinant SgaMan1 polypeptides, compositions and methods comprising these variants are not substantially identical to SEQ ID NO:2 or SEQ ID NO:3, but rather include amino acid substitutions, insertions, or deletions that affect, in certain circumstances, substantially, the structure, function, or expression of the polypeptide herein such that improved characteristics, including, e.g., improved specific activity to hydrolyze a mannan-containing lignocellulosic substrate, more rapid viscosity reduction when used to treat high solids biomass substrates, improved expression in a desirable host organism, improved thermostability, pH stability, etc, as compared to that of a polypeptide of SEQ ID NO:2 or SEQ ID NO:3 can be achieved.

In some embodiments, the recombinant SgaMan1 polypeptide (including a variant thereof) has beta-mannanase activity. Beta-mannanase activity can be determined using an assay measuring the release of reducing sugars from a galactomannan substrate, for example, in accordance with the description of Example 5. Beta-mannanase activity can be determined by combining with a cellulase and/or hemicellulase mixture, followed by using such a mixture to treat a suitable mannan-containing biomass substrate, such as, for example, a woody substrate, etc., in accordance with the protocols and conditions described in, for example, Example 9, or by suitable assays, or methods of activity measurement known in the art.

Recombinant SgaMan1 polypeptides include fragments of “full-length” SgaMan1 polypeptides that retain beta-mannanase activity. Preferably those functional fragments (i.e., fragments that retain beta-mannanase activity) are at least 80 amino acid residues in length (e.g., at least 80 amino acid residues, at least 100 amino acid residues, at least 120 amino acid residues, at least 140 amino acid residues, at least 160 amino acid residues, at least 180 amino acid residues, at least 200 amino acid residues, at least 250 amino acid residues, at least 300 amino acid residues, at least 350 amino acid residues, at least 400 amino acid residues, or even at least 450 amino acid residues in length or longer). Such fragments suitably retain the active site of the full-length precursor polypeptides or full length mature polypeptides but may have deletions of non-critical amino acid residues. The activity of fragments can be readily determined using the methods of measuring beta-mannanase activity described herein, for example the assay described in Example 5, and the hydrolysis performance measurements as those described in Example 9, or by suitable assays or other means of activity measurements known in the art.

In some embodiments, the SgaMan1 amino acid sequences and derivatives are produced as an N- and/or C-terminal fusion protein, for example, to aid in extraction, detection and/or purification and/or to add functional properties to the SgaMan1 polypeptides. Examples of fusion protein partners include, but are not limited to, glutathione-S-transferase (GST), 6× His, GAL4 (DNA binding and/or transcriptional activation domains), FLAG-, MYC-tags or other tags known to those skilled in the art. In some embodiments, a proteolytic cleavage site is provided between the fusion protein partner and the polypeptide sequence of interest to allow removal of fusion sequences. Suitably, the fusion protein does not hinder the activity of the recombinant SgaMan1 polypeptide. In some embodiments, the recombinant SgaMan1 polypeptide is fused to a functional domain including a leader peptide, propeptide, binding domain and/or catalytic domain. Fusion proteins are optionally linked to the recombinant SgaMan1 polypeptide through a linker sequence that joins the SgaMan1 polypeptide and the fusion domain without significantly affecting the properties of either component. The linker optionally contributes functionally to the intended application.

The present disclosure provides host cells that are engineered to express one or more SgaMan1 polypeptides of the disclosure. Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus licheniformis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus,Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

Methods of transforming nucleic acids into these organisms are known in the art. For example, a suitable procedure for transforming Aspergillus host cells is described in EP 238 023.

In some embodiments, the recombinant SgaMan1 polypeptide is fused to a signal peptide to, for example, facilitate extracellular secretion of the recombinant SgaMan1 polypeptide. For example, in certain embodiments, the signal peptide is a non-native signal peptide such as the B. subtilis AprE signal peptide of SEQ ID NO:9. In some embodiments, the SgaMan1 polypeptide has an N-terminal extension of Ala-Gly-Lys between the mature form and the signal polypeptide. In particular embodiments, the recombinant SgaMan1 polypeptide is expressed in a heterologous organism as a secreted polypeptide. The compositions and methods herein thus encompass methods for expressing a SgaMan1 polypeptide as a secreted polypeptide in a heterologous organism.

The disclosure also provides expression cassettes and/or vectors comprising the above-described nucleic acids. Suitably, the nucleic acid encoding a SgaMan1 polypeptide of the disclosure is operably linked to a promoter. Promoters are well known in the art. Any promoter that functions in the host cell can be used for expression of a beta-mannanase and/or any of the other nucleic acids of the present disclosure. Initiation control regions or promoters, which are useful to drive expression of a beta-mannanase nucleic acids and/or any of the other nucleic acids of the present disclosure in various host cells are numerous and familiar to those skilled in the art (see, for example, WO 2004/033646 and references cited therein). Virtually any promoter capable of driving these nucleic acids can be used.

Specifically, where recombinant expression in a filamentous fungal host is desired, the promoter can be a filamentous fungal promoter. The nucleic acids can be, for example, under the control of heterologous promoters. The nucleic acids can also be expressed under the control of constitutive or inducible promoters. Examples of promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or beta-glucosidase promoter. A particulary suitable promoter can be, for example, a T. reesei cellobiohydrolase, endoglucanase, or beta-glucosidase promoter. For example, the promoter is a cellobiohydrolase I (cbh1) promoter. Non-limiting examples of promoters include a cbh1, cbh2, egl1, egl2, egl3, egl4, egl5, pki1, gpd1, xyn1, or xyn2 promoter. Additional non-limiting examples of promoters include a T. reesei cbh1, cbh2, egl1, egl2, egl3, egl4, egl5, pki1, gpd1, xyn1, or xyn2 promoter.

The nucleic acid sequence encoding a SgaMan1 polypeptide herein can be included in a vector. In some aspects, the vector contains the nucleic acid sequence encoding the SgaMan1 polypeptide under the control of an expression control sequence. In some aspects, the expression control sequence is a native expression control sequence. In some aspects, the expression control sequence is a non-native expression control sequence. In some aspects, the vector contains a selective marker or selectable marker. In some aspects, the nucleic acid sequence encoding the SgaMan1 polypeptide is integrated into a chromosome of a host cell without a selectable marker.

Suitable vectors are those which are compatible with the host cell employed. Suitable vectors can be derived, for example, from a bacterium, a virus (such as bacteriophage T7 or a M-13 derived phage), a cosmid, a yeast, or a plant. Suitable vectors can be maintained in low, medium, or high copy number in the host cell. Protocols for obtaining and using such vectors are known to those in the art (see, for example, Sambrook et al., Molecular Cloning: A Laboratory Manual, 2^{nd} ed., Cold Spring Harbor, 1989).

In some aspects, the expression vector also includes a termination sequence. Termination control regions may also be derived from various genes native to the host cell. In some aspects, the termination sequence and the promoter sequence are derived from the same source.

A nucleic acid sequence encoding a SgaMan1 polypeptide can be incorporated into a vector, such as an expression vector, using standard techniques (Sambrook et al., Molecular Cloning: A Laboratory Manual, Cold Spring Harbor, 1982).

In some aspects, it may be desirable to over-express a SgaMan1 polypeptide and/or one or more of any other nucleic acid described in the present disclosure at levels far higher than currently found in naturally-occurring cells. In some embodiments, it may be desirable to under-express (e.g., mutate, inactivate, or delete) an endogenous beta-mannanase and/or one or more of any other nucleic acid described in the present disclosure at levels far below that those currently found in naturally-occurring cells.

### SgaMan1 -Encoding Polynucleotides

Another aspect of the compositions and methods described herein is a polynucleotide or a nucleic acid sequence that encodes a recombinant SgaMan1 polypeptide (including variants and fragments thereof) having beta-mannanase activity. In some embodiments the polynucleotide is provided in the context of an expression vector for directing the expression of a SgaMan1 polypeptide in a heterologous organism, such as one identified herein. The polynucleotide that encodes a recombinant SgaMan1 polypeptide may be operably-linked to regulatory elements (e.g., a promoter, terminator, enhancer, and the like) to assist in expressing the encoded polypeptides.

An example of a polynucleotide sequence encoding a recombinant SgaMan1 polypeptide has the nucleotide sequence of SEQ ID NO: 1. Similar, including substantially identical, polynucleotides encoding recombinant SgaMan1 polypeptides and variants may occur in nature, e.g., in other strains or isolates of Streptococcus gallolyticus, or Streptococcus sp. In view of the degeneracy of the genetic code, it will be appreciated that polynucleotides having different nucleotide sequences may encode the same SgaMan1 polypeptides, variants, or fragments.

In some embodiments, polynucleotides encoding recombinant SgaMan1 polypeptides have a specified degree of amino acid sequence identity to the exemplified polynucleotide encoding a SgaMan1 polypeptide, e.g., at least 55%, at last 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% sequence identity to the amino acid sequence of SEQ ID NO: 2, or to the mature sequence of SEQ ID NO:3. Homology can be determined by amino acid sequence alignment, e.g., using a program such as BLAST, ALIGN, or CLUSTAL, as described herein.

In some embodiments, the polynucleotide that encodes a recombinant SgaMan1 polypeptide is fused in frame behind (i.e., downstream of) a coding sequence for a signal peptide for directing the extracellular secretion of a recombinant SgaMan1 polypeptide. As described herein, the term “heterologous” when used to refer to a signal sequence used to express a polypeptide of interest, it is meant that the signal sequence and the polypeptide of interest are from different organisms. Heterologous signal sequences include, for example, those from other fungal cellulase genes, such as, e.g., the signal sequence of Trichoderma reesei CBH1. Expression vectors may be provided in a heterologous host cell suitable for expressing a recombinant SgaMan1 polypeptide, or suitable for propagating the expression vector prior to introducing it into a suitable host cell.

In some embodiments, polynucleotides encoding recombinant SgaMan1 polypeptides hybridize to the polynucleotide of SEQ ID NO:1 (or to the complement thereof) under specified hybridization conditions. Examples of conditions are intermediate stringency, high stringency and extremely high stringency conditions, which are described herein.

SgaMan1 polynucleotides may be naturally occurring or synthetic (i.e., man-made), and may be codon-optimized for expression in a different host, mutated to introduce cloning sites, or otherwise altered to add functionality.

The nucleic acid sequence encoding the coding region of SgaMan1 polypeptide derived from Streptococcus gallolyticus UCN34 is as follows (SEQ ID NO: 1):

ATGAAAAAAGCATTAGTTGGATCTTTAGCGACCCTAACTGTCGTTGCCG
GTCTCGCATCAGCTCAAGGGGTAAAAGCTGACGAAATCGTTTCAGGACA
GACTTACAAAATCACTGCAAAACATAGTGGTAAAGCTCTTGACGTTGCA
TCAAAAGCAACTTACGCAGGAGCTAACGTGCAACAATGGTCTTATAACG
GCGGCACTAACCAACAATGGAAAGTTGTTGATACAGGAGACGGATACTA
CAAATTAGTATCAGTAAACAGTGGTAAAGTGCTAGACGTAGCAAGTGCA
GCAACTTACAATGGTGCTAACGTACAACAATGGGACGATACTAACGGAA
CATGCCAACGCTGGAAAATCGTAGATACTGGTGACGGTTCTTACAAACT
AATCTCTGCTGTCAGCGGTAAAGCTCTTGACGTTGCAGCAAACTCTAGC
GATGACGGTGCCAACGTACAACAATGGGATGACAACGGTGGTTGGAACC
AAAAATGGGAATTCACAAAAGTAAGTGACAGTGGAAACACTGGTGGTGA
TACTGGTGGCGACACTACTACTCCAGTTTCAACTAGTGGTTTCTACACA
AACGGTACTACACTTTATGACGCAAAAGGTAATCCATTTGTGATGCGCG
GTATCAACCACGCTCACTCTTGGTACCAAGGTTATGAAGATACTGCTAT
TCCTGCCATTGCAAAAACTGGCGCTAACTGTGTTCGCTTAGTTATCACA
GATGGTCAACAATACAGTAAAACTTCACTCTCTGAACTTCAAAAACTTA
TTCAACTTTGTAAAGATAACAAATTAATTGCTATCGTTGAAGTGCATGA
CGCTACTGGTTCAGATAACATTCAAGACTTAGAAAAAGCTGCTGATTAC
TGGATTGAAATGAAGAGTGCTTTGATTGGAAACGAAGCAAACGTCATCC
TTAACATCGCTAACGAATGGGGCGGTGCTTGGGACAGTTCAAACTGGGC
AGCAGGTTACCAACAAGTTATTCCAAAACTTCGTAATGCTGGTATCAAA
AACACACTTATGGTCGATTGTGCTGGTTGGGGACAATACCCACAATCTA
TCGCAGAAGCAGGTCAAAGTGTCGTTCAAGCTGATTCACAACACAACAT
CATGTTCTCTATTCACATGTACGAATATGCTGGTGCATACGGCGCAGTT
CAATCAAACATTGACTCTGCACTTAACGTAGGTGTACCACTTTGTATCG
GTGAATTCGGTATCCGACACACTAACGGCGATGTAGATTACAAGACAAT
CATGAGCTACTCTCAATCTAAGGGAGTTGGTTACCTCGGTTGGTCTTGG
AAAGGTAACGGCGACACTTGGGCTTACCTCGATATCGCACAAGATTGGA
GCGGTTCACAATTAACTGAACAAGGTAACGCTATTATCTATGACCAAAA
TGGTATCAAGAACACATCTAAACGTTGTACAATTTTTGACTAA

As is well known to those of ordinary skill in the art, due to the degeneracy of the genetic code, polynucleotides having significantly different sequences can nonetheless encode identical, or nearly identical, polypeptides. As such, aspects of the present compositions and methods include polynucleotides encoding SgaMan1 polypeptides or derivatives thereof that contain a nucleic acid sequence that is at least 55% identical to SEQ ID NO: 1, including at least 55%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:1. In some embodiments, SgaMan1 polypeptides contain a nucleic acid sequence that is identical to SEQ ID NO: 1.

In some embodiments, polynucleotides may include a sequence encoding a signal peptide. Many convenient signal sequences may be suitably employed.

Purification from Natural Isolates

The SgaMan1 polypeptides can be purified from natural isolates (e.g., from a strain of Streptococcus gallolyticus) by known and commonly employed methods. For example, cells containing a SgaMan1 polypeptide can be disrupted by various physical or chemical means, such as freeze-thaw cycling, sonication, mechanical disruption, or cell lysing agents. Cell supernatants may be collected (for example from cells that secrete the protein into the medium). The SgaMan1 polypeptide can be recovered from the medium and/or lysate by conventional techniques including separations of the cells/debris from the medium by centrifugation, filtration, and precipitation of the proteins in the supernatant or filtrate with a salt, for example, ammonium sulphate. The SgaMan1 polypeptide can then be purified from the disrupted cells by procedures such as: fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and affinity chromatography. Various methods of protein purification may be employed and such methods are known in the art and described for example in Deutscher, Methods in Enzymology, 182 (1990); Scopes, Protein Purification: Principles and Practice, Springer-Verlag, New York (1982).

### Chemical Synthesis

Alternatively, the SgaMan1 polypeptide sequence, or portions thereof, may be produced by direct peptide synthesis using solid-phase techniques (see, e.g., Stewart et al., Solid-Phase Peptide Synthesis, W. H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of SgaMan1 may be chemically synthesized separately and combined using chemical or enzymatic methods to produce a full-length SgaMan1.

### Recombinant Methods of Making

### Isolation of DNA Encoding the SgaMan1 Polypeptide

DNA encoding a SgaMan1 polypeptide may be obtained from a cDNA library prepared from a microorganism believed to possess the SgaMan1 mRNA (e.g., Streptococcus gallolyticus) and to express it at a detectable level. The SgaMan1 -encoding gene may also be obtained from a genomic library or by oligonucleotide synthesis.

Libraries can be screened with probes (such as antibodies to a SgaMan1 or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding a SgaMan1 is to use PCR methodology (Sambrook et al., supra; Dieffenbach et al., PCR Primer:A Laboratory Manual (Cold Spring Harbor Laboratory Press, 1995)).

In known techniques for screening a cDNA library, the oligonucleotide sequences selected as probes should be of sufficient length and sufficiently unambiguous that false positives are minimized. The oligonucleotide can be labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., Molecular Cloning: A Laboratory Manual (New York: Cold Spring Harbor Laboratory Press, 1989).

Nucleic acids having protein coding sequence may be obtained by screening selected cDNA or genomic libraries using the deduced amino acid sequence disclosed herein for the first time, and, if necessary, using conventional primer extension procedures as described in Sambrook et al., Molecular Cloning: A Laboratory Manual (New York: Cold Spring Harbor Laboratory Press, 1989), to detect precursors and processing intermediates of mRNA that may not have been reverse-transcribed into cDNA.

### Selection and Transformation of Host Cells

Host cells are transfected or transformed with expression or cloning vectors described herein for SgaMan1 production. The host cells are cultured in conventional nutrient media modified as appropriate for inducing promoters, selecting transformants, or amplifying the genes encoding the desired sequences. The culture conditions, such as media, temperature, pH and the like, can be selected by the ordinarily skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., Molecular Cloning: A Laboratory Manual (New York: Cold Spring Harbor Laboratory Press, 1989).

Methods of transfection are known to the ordinarily skilled artisan, for example, CaPO₄ and electroporation. Depending on the host cell used, transformation is performed using standard techniques appropriate to such cells. The calcium treatment employing calcium chloride, as described in Sambrook et al., Molecular Cloning: A Laboratory Manual (New York: Cold Spring Harbor Laboratory Press, 1989), or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. Transformations into yeast can be carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, microporation, biolistic bombardment, bacterial protoplast fusion with intact cells, or polycations, e.g., polybrene, polyornithine, may also be used.

Suitable host cells for cloning or expressing the DNA in the vectors herein include prokaryote, yeast, or filamentous fungal cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). In addition to prokaryotes, eukaryotic microorganisms such as filamentous fungi or yeast are suitable cloning or expression hosts for vectors encoding SgaMan1 polypeptides. Saccharomyces cerevisiae is a commonly used lower eukaryotic host microorganism.

In some embodiments, the microorganism to be transformed includes a strain derived from Trichoderma spp. or Aspergillus spp. Exemplary strains include T. reesei which is useful for obtaining overexpressed protein or Aspergillus niger var. awamori. For example, Trichoderma strain RL-P37, described by Sheir-Neiss et al. in Appl. Microbiol. Biotechnology, 20 (1984) pp. 46-53 is known to secrete elevated amounts of cellulase enzymes. Functional equivalents of RL-P37 include Trichoderma reesei (longibrachiatum) strain RUT-C30 (ATCC No. 56765) and strain QM9414 (ATCC No. 26921). Another example includes overproducing mutants as described in Ward et al. in Appl. Microbiol. Biotechnology 39:738-743 (1993). For example, it is contemplated that these strains would also be useful in overexpressing a Streptococcus gallolyticus SgaMan1 polypeptide, or a variant thereof. The selection of the appropriate host cell is deemed to be within the skill in the art.

### Preparation and Use of a Replicable Vector

DNA encoding the SgaMan1 protein or derivatives thereof (as described above) is prepared for insertion into an appropriate microorganism. According to the present compositions and methods, DNA encoding a SgaMan1 polypeptide includes all of the DNA necessary to encode for a protein which has functional SgaMan1 activity. As such, embodiments of the present compositions and methods include DNA encoding a SgaMan1 polypeptide derived from Streptococcus spp., including, Streptococcus gallolyticus, such as Streptococcus gallolyticus UCN34.

The DNA encoding SgaMan1 may be prepared by the construction of an expression vector carrying the DNA encoding SgaMan1. The expression vector carrying the inserted DNA fragment encoding the SgaMan1 may be any vector which is capable of replicating autonomously in a given host organism or of integrating into the DNA of the host, typically a plasmid, cosmid, viral particle, or phage. Various vectors are publicly available. It is also contemplated that more than one copy of DNA encoding a SgaMan1 may be recombined into the strain to facilitate overexpression.

In certain embodiments, DNA sequences for expressing SgaMan1 include the promoter, gene coding region, and terminator sequence all originate from the native gene to be expressed. Gene truncation may be obtained by deleting away undesired DNA sequences (e.g., coding for unwanted domains) to leave the domain to be expressed under control of its native transcriptional and translational regulatory sequences. A selectable marker can also be present on the vector allowing the selection for integration into the host of multiple copies of the SgaMan1 gene sequences.

In other embodiments, the expression vector is preassembled and contains sequences required for high level transcription and, in some cases, a selectable marker. It is contemplated that the coding region for a gene or part thereof can be inserted into this general purpose expression vector such that it is under the transcriptional control of the expression cassette's promoter and terminator sequences. For example, pTEX is such a general purpose expression vector. Genes or part thereof can be inserted downstream of the strong cbh1 promoter.

In the vector, the DNA sequence encoding the SgaMan1 of the present compositions and methods should be operably linked to transcriptional and translational sequences, e.g., a suitable promoter sequence and signal sequence in reading frame to the structural gene. The promoter may be any DNA sequence which shows transcriptional activity in the host cell and may be derived from genes encoding proteins either homologous or heterologous to the host cell. The signal peptide provides for extracellular production (secretion) of the SgaMan1 or derivatives thereof. The DNA encoding the signal sequence can be that which is naturally associated with the gene to be expressed. However the signal sequence from any suitable source, for example an exo-cellobiohydrolases or endoglucanase from Trichoderma, a xylanase from a bacterial species, e.g., from Streptomyces coelicolor, etc., are contemplated in the present compositions and methods.

The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

A desired SgaMan1 polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector or it may be a part of the SgaMan1-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990.

Both expression and cloning vectors may contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria and the 2μ plasmid origin is suitable for yeast.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase forBacilli. A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)). An exemplary selection gene for use in Trichoderma sp is the pyr4 gene.

Expression and cloning vectors usually contain a promoter operably linked to the SgaMan1 -encoding nucleic acid sequence. The promoter directs mRNA synthesis. Promoters recognized by a variety of potential host cells are well known. Promoters include a fungal promoter sequence, for example, the promoter of the cbh1 or egl1 gene.

Promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Additional promoters, e.g., the A4 promoter from A. niger, also find use in bacterial expression systems, e.g., in S. lividans. Promoters for use in bacterial systems also may contain a Shine-Dalgarno (S.D.) sequence operably linked to the DNA encoding a SgaMan1 polypeptide.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Expression vectors used in eukaryotic host cells (e.g. yeast, fungi, insect, plant) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding a SgaMan1 polypeptide.

### Purification of a SgaMan1 Polypeptide

Forms of SgaMan1 polypeptides (or SgaMan1 polypeptide derivatives) may be recovered from culture medium or from host cell lysates by the methods described above for isolation and purification from natural isolates. Additional techniques can be used depending on the host cell employed and any variant structures in the recombinant enzyme. For example, if the recombinant enzyme is membrane-bound, it can be released from the membrane using a suitable detergent solution (e.g. Triton-X 100) or by enzymatic cleavage. Purification of recombinant enzyme may also employ protein A Sepharose columns to remove contaminants such as IgG and metal chelating columns to bind epitope-tagged forms of the SgaMan1 polypeptide. The purification step(s) selected will depend, for example, on the nature of the production process used, the particular SgaMan1 polypeptide that is produced, and any variant structure for the recombinant enzyme. Antibodies directed to a SgaMan1 polypeptide or epitope tags thereon may also be employed to purify the protein, e.g., anti-SgaMan1 antibodies attached to a solid support.

### Derivatives of SgaMan1

As described above, in addition to the native sequence of SgaMan1 described herein (e.g., as depicted in full length as SEQ ID NO:2, and in the mature form as SEQ ID NO: 3), it is contemplated that SgaMan1 derivatives can be prepared with altered amino acid sequences. In general, SgaMan1 derivatives would be capable of conferring, as a native SgaMan1 polypeptide, to a cellulase and/or hemicellulase mixture or composition either one or both of an improved capacity to hydrolyze a lignocellulosic biomass substrate, in particular one that is mannan-containing, and an improved capacity to reduce viscosity of a biomass substrate mixture, particularly one that is at a high solids level. Such derivatives may be made, for example, to improve expression in a particular host, improve secretion (e.g., by altering the signal sequence), to introduce epitope tags or other sequences that can facilitate the purification and/or isolation of SgaMan1 polypeptides. In some embodiments, derivatives may confer more capacity to hydrolyze a lignocellulosic biomass substrate to a cellulase and/or hemicellulase mixture or compostion, as compared to the native SgaMan1 polypeptide. In some embodiments, derivatives may confer a higher viscosity reduction benefit (e.g., an improvement or even higher speed and/or extent of viscosity reduction) to a cellulase and/or hemicellulase mixture, as compared to the native SgaMan1 polypeptide.

SgaMan1 polypeptide derivatives can be prepared by introducing appropriate nucleotide changes into the SgaMan1 -encoding DNA, or by synthesis of the desired SgaMan1 polypeptides. Those skilled in the art will appreciate that amino acid changes may alter post-translational processes of the SgaMan1 polypetpides, such as changing the number or position of glycosylation sites.

Derivatives of the native sequence SgaMan1 polypeptide or of various domains of the SgaMan1 described herein can be made, for example, using any of the techniques and guidelines for conservative and non-conservative mutations set forth, for instance, in U.S. Pat. No. 5,364,934. Sequence variations may be a substitution, deletion or insertion of one or more codons encoding the SgaMan1 polypeptide that results in a change in the amino acid sequence of the SgaMan1 polypeptide as compared with the native sequence SgaMan1 polypeptide. Optionally, the sequence variation is by substitution of at least one amino acid with any other amino acid in one or more of the domains of the SgaMan1 polypeptide.

Guidance in determining which amino acid residue may be inserted, substituted or deleted without adversely affecting the desired SgaMan1 beta-mannanase activity may be found by comparing the sequence of the polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting derivatives for functional activity using techniques known in the art.

The sequence variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the SgaMan1 -encoding DNA with a variant sequence.

Scanning amino acid analysis can also be employed to identify one or more amino acids along a contiguous sequence. Among the scanning amino acids the can be employed are relatively small, neutral amino acids. Such amino acids include alanine, glycine, serine, and cysteine. Alanine is often used as a scanning amino acid among this group because it eliminates the side-chain beyond the beta-carbon and is less likely to alter the main-chain conformation of the derivative. Alanine is also often used because it is the most common amino acid. Further, it is frequently found in both buried and exposed positions (Creighton, The Proteins, (W. H. Freeman & Co., N.Y.); Chothia, J. Mol. Biol., 150:1 (1976)). If alanine substitution does not yield adequate amounts of derivative, an isosteric amino acid can be used.

### Anti-SgaMan1 Antibodies

The present compositions and methods further provides anti-SgaMan1 antibodies. Exemplary antibodies include polyclonal and monoclonal antibodies, including chimeric and humanized antibodies.

The anti-SgaMan1 antibodies of the present compositions and methods may include polyclonal antibodies. Any convenient method for generating and preparing polyclonal and/or monoclonal antibodies may be employed, a number of which are known to those ordinarily skilled in the art.

Anti-SgaMan1 antibodies may also be generated using recombinant DNA methods, such as those described in U.S. Pat. No. 4,816,567.

The antibodies may be monovalent antibodies, which may be generated by recombinant methods or by the digestion of antibodies to produce fragments thereof, particularly, Fab fragments.

### Cell Culture Media

Generally, the microorganism is cultivated in a cell culture medium suitable for production of the SgaMan1 polypeptides described herein. The cultivation takes place in a suitable nutrient medium comprising carbon and nitrogen sources and inorganic salts, using procedures and variations known in the art. Suitable culture media, temperature ranges and other conditions for growth and cellulase production are known in the art. As a non-limiting example, a typical temperature range for the production of cellulases by Trichoderma reesei is 24° C. to 37° C., for example, between 25° C. and 30° C.

### Cell Culture Conditions

Materials and methods suitable for the maintenance and growth of fungal cultures are well known in the art. In some aspects, the cells are cultured in a culture medium under conditions permitting the expression of one or more beta-mannanase polypeptides encoded by a nucleic acid inserted into the host cells. Standard cell culture conditions can be used to culture the cells. In some aspects, cells are grown and maintained at an appropriate temperature, gas mixture, and pH. In some aspects, cells are grown at in an appropriate cell medium.

### Compositions Comprising a Recombinant Beta-Mannanase SgaMan1 Polypeptide

The present disclosure provides engineered enzyme compositions (e.g., cellulase compositions) or fermentation broths enriched with a recombinant SgaMan1 polypeptides. In some aspects, the composition is a cellulase composition. The cellulase composition can be, e.g., a filamentous fungal cellulase composition, such as a Trichoderma cellulase composition. The cellulase composition can be, in some embodiments, an admixture or physical mixture, of various cellulases originating from different microorganisms; or it can be one that is the culture broth of a single engineered microbe co-expressing the celluase genes; or it can be one that is the admixture of one or more individually/separately obtained cellulases with a mixture that is the culture broth of an engineered microbe co-expressing one or more cellulase genes.

In some aspects, the composition is a cell comprising one or more nucleic acids encoding one or more cellulase polypeptides. In some aspects, the composition is a fermentation broth comprising cellulase activity, wherein the broth is capable of converting greater than about 50% by weight of the cellulose present in a biomass sample into sugars. The term “fermentation broth” and “whole broth” as used herein refers to an enzyme preparation produced by fermentation of an engineered microorganism that undergoes no or minimal recovery and/or purification subsequent to fermentation. The fermentation broth can be a fermentation broth of a filamentous fungus, for example, a Trichoderma, Humicola, Fusarium, Aspergillus, Neurospora, Penicillium, Cephalosporium, Achlya, Podospora, Endothia, Mucor, Cochliobolus, Pyricularia, Myceliophthora or Chrysosporium fermentation broth. In particular, the fermentation broth can be, for example, one of Trichoderma spp. such as a Trichoderma reesei, or Penicillium spp., such as a Penicillium funiculosum. The fermentation broth can also suitably be a cell-free fermentation broth. In one aspect, any of the cellulase, cell, or fermentation broth compositions of the present invention can further comprise one or more hemicellulases.

In some aspects, the whole broth composition is expressed in T. reesei or an engineered strain thereof In some aspects the whole broth is expressed in an integrated strain of T. reesei wherein a number of cellulases including a SgaMan1 polypeptide has been integrated into the genome of the T. reesei host cell. In some aspects, one or more components of the polypeptides expressed in the integrated T. reesei strain have been deleted.

In some aspects, the whole broth composition is expressed in A. niger or an engineered strain thereof.

Alternatively, the recombinant SgaMan1 polypeptides can be expressed intracellularly. Optionally, after intracellular expression of the enzyme variants, or secretion into the periplasmic space using signal sequences such as those mentioned above, a permeabilisation or lysis step can be used to release the recombinant SgaMan1 polypeptide into the supernatant. The disruption of the membrane barrier is effected by the use of mechanical means such as ultrasonic waves, pressure treatment (French press), cavitation, or by the use of membrane-digesting enzymes such as lysozyme or enzyme mixtures.

In some aspects, the polynucleotides encoding the recombinant SgaMan1 polypeptide are expressed using a suitable cell-free expression system. In cell-free systems, the polynucleotide of interest is typically transcribed with the assistance of a promoter, but ligation to form a circular expression vector is optional. In some embodiments, RNA is exogenously added or generated without transcription and translated in cell-free systems.

### Uses of SgaMan1 Polypeptides to Hydrolyze a Lignocellulosic Biomass Substrate

In some aspects, provided herein are methods for converting lignocelluloses biomass to sugars, the method comprising contacting the biomass substrate with a composition disclosed herein comprising a SgaMan1 polypeptide in an amount effective to convert the biomass substrate to fermentable sugars. Suitably the biomass substrate comprises GGM and/or GM. In certain embodiments, a suitable biomass substrate may contain up to about 2 wt. % or more, about 3 wt. % or more, about 4 wt. % or more, about 5 wt. % or more, etc. of GGM and/or GM.

In some aspects, the method further comprises pretreating the biomass with acid and/or base and/or mechanical or other physical means In some aspects the acid comprises phosphoric acid. In some aspects, the base comprises sodium hydroxide or ammonia. In some aspects, the mechanical means may include, for example, pulling, pressing, crushing, grinding, and other means of physically breaking down the lignocellulosic biomass into smaller physical forms. Other physical means may also include, for example, using steam or other pressurized fume or vapor to “loosen” the lignocellulosic biomass in order to increase accessibility by the enzymes to the cellulose and hemicellulose. In certain embodiments, the method of pretreatment may also involve enzymes that are capable of breaking down the lignin of the lignocellulosic biomass substrate, such that the accessibility of the enzymes of the biomass hydrolyzing enzyme composition to the cellulose and the hemicelluloses of the biomass is increased.

Biomass: The disclosure provides methods and processes for biomass saccharification, using the enzyme compositions of the disclosure, comprising a SgaMan1 polypeptide. The term “biomass,” as used herein, refers to any composition comprising cellulose and/or hemicellulose (optionally also lignin in lignocellulosic biomass materials). Particularly suitable are lignocellulosic biomass materials comprising measureable amounts of galactoglucomannans (GGMs) and/or glucomannan (GMs). Such biomass materials may include, for example, a KRAFT-alkaline pretreated industrial unbleached softwood pulp, FPP-27, which can be obtained from Agence Nationale de la Recherche, France, which contains about 6.5 wt. % mannan; a SPORL-pretreated softwood (Zhu J. Y. et al., (2010) Appl. Microbiol. Biotechnol. 86(5):1355-65; Tian S. et al., (2010) Bioresour. Technol. 101:8678-85), which contains about 4.5 wt. % mannan; spruce, which may contain over 10 wt. % of mannan. As used herein, biomass includes, without limitation, certain softwood trees such as spruce, pine, aspen trees, and wastes derived therefrom, seeds, grains, tubers, plant waste (such as, for example, empty fruit bunches of the palm trees, or palm fibre wastes) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The disclosure therefore provides methods of saccharification comprising contacting a composition comprising a biomass material, for example, a material comprising xylan, hemicellulose, and in particular, galactoglucomannans (GGMs) and/or glucomannans (GMs), cellulose, and/or a fermentable sugar, with a SgaMan1 polypeptide of the disclosure, or a SgaMan1 polypeptide encoded by a nucleic acid or polynucleotide of the disclosure, or any one of non-naturally occurring the cellulase and/or hemicellulase compositions comprising a SgaMan1 polypeptide, or products of manufacture of the disclosure.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Pretreatment: Prior to saccharification or enzymatic hydrolysis and/or fermentation of the fermentable sugars resulting from the saccharifiction, biomass (e.g., lignocellulosic material) is preferably subject to one or more pretreatment step(s) in order to render xylan, hemicellulose, cellulose and/or lignin material more accessible or susceptible to the enzymes in the enzymatic composition (for example, the enzymatic composition of the present invention comprising a SgaMan1 polypeptide) and thus more amenable to hydrolysis by the enzyme(s) and/or the enzyme compositions.

In some aspects, a suitable pretreatment method may involve subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

In some aspects, a suitable pretreatment method may involve subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

In further aspects, a suitable pretreatment method may involve processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841.

In yet further aspects, a suitable pretreatment method may involve pre-hydrolyzing biomass (e.g., lignocellulosic materials) in a pre-hydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat No. 5,705,369. In a variation of this aspect, the pre-hydrolyzing can alternatively or further involves pre-hydrolysis using enzymes that are, for example, capable of breaking down the lignin of the lignocellulosic biomass material.

In yet further aspects, suitable pretreatments may involve the use of hydrogen peroxide H₂O₂. See Gould, 1984, Biotech, and Bioengr. 26:46-52.

In further aspects, suitable pretreatment of the lignocellulosic biomass materials, in particular those comprising measurable amounts of galactoglucomannans (GGMs) and/or glucomannans (GMs), may include the KRAFT alkaline pretreatment method employed by, for example, the Agence Nationale de la Recherche, France. The KRAFT pretreatment method is a well-known and widely used method to convert wood into wood pulp, typically including the treatment of wood chips with a mixture of sodium hydroxide and sodium sulfide, known in the industry as “white liquor,” which breaks down the bonds that link lignin to the cellulose. It is a long-practiced method, mostly in the paper and pulp industry, originally invented by Carl F. Dahl in 1879, as described in U.S. Pat. 296,935, issued in 1884. Also included are the SPORL pretreatment method developed by the United States Department of Agriculture specifically for certain softwood biomass feedstocks, for example, for pine, spruce and aspen tree materials, such as described in Zhu et al., (2009) Bioresource Technol. 100:2411-18. The SPORL pretreatment method involves using sulfite to treat wood chips of such softwoods under acidic conditions followed by mechanical size reduction using disk refining. The SPORL method was reported to produce reduced amounts of fermentation inhibitors such as hydroxyl-methyl furfural and/or furfural.

In other aspects, pretreatment can also comprise contacting a biomass material with stoichiometric amounts of sodium hydroxide and ammonium hydroxide at a very low concentration. See Teixeira et al., (1999), Appl. Biochem.and Biotech. 77-79:19-34.

In some embodiments, pretreatment can comprise contacting a lignocellulose with a chemical (e.g., a base, such as sodium carbonate or potassium hydroxide) at a pH of about 9 to about 14 at moderate temperature, pressure, and pH. See Published International Application WO2004/081185. Ammonia is used, for example, in a preferred pretreatment method. Such a pretreatment method comprises subjecting a biomass material to low ammonia concentration under conditions of high solids. See, e.g., U.S. Patent Publication No. 20070031918 and Published International Application WO 06110901.

### The Saccharification Process

In some aspects, provided herein is a saccharification process comprising treating a lignocellulosic biomass material, in particular, one comprising a measurable amount of galactoglucomannans (GGMs) and/or glucomannans (GMs), with an enzyme composition comprising a polypeptide, wherein the polypeptide has beta-mannanase activity and wherein the process results in at least about 50 wt. % (e.g., at least about 55 wt. %, 60 wt. %, 65 wt. %, 70 wt. %, 75 wt. %, or 80 wt. %) conversion of the biomass to fermentable sugars. In some aspects, the biomass comprises lignin. In some aspects the biomass comprises cellulose. In some aspects the biomass comprises hemicelluloses. In some aspects, the biomass comprising cellulose further comprises one or more of mannan, xylan, galactan, and/or arabinan. In certain particular aspects, the biomass comprising cellulose as well as at least a measurable level of galactoglucomannan and/or glucomannan. In some aspects, the biomass may be, without limitation, softwood plants (e.g., pine, spruce, aspen trees), seeds, grains, tubers, plant waste (e.g., empty fruit bunch from palm trees, or palm fibre waste) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), woody materials (including, e.g., wood chips, processing waste), paper, pulp, and recycled paper (including, e.g., newspaper, printer paper, and the like), potatoes, soybean (e.g., rapeseed), barley, rye, oats, wheat, beets, and sugar cane bagasse.

In some aspects, the material comprising biomass is subject to one or more pretreatment methods/steps prior to treatment with the SgaMan1 polypeptide or the composition comprising the SgaMan1 polypeptide. In some aspects, the saccharification or enzymatic hydrolysis further comprises treating the biomass with an enzyme composition comprising a SgaMan1 polypeptide of the invention. The enzyme composition may, for example, comprise one or more cellulases, for example, one or more endoglucanases, one or more cellobiohydrolases, and/or one or more beta-glucosidases, in addition to the SgaMan1 polypeptide. Alternatively, the enzyme composition may comprise one or more other hemicellulases, for example, one or more other beta-mannanases, one or more xylanases, one or more beta-xylosidases, and/or one or more L-arabinofuranosidases. In certain embodiments, the enzyme composition comprises a SgaMan1 polypeptide of the invention, one or more cellulases, one or more other hemicellulases. In some embodiments, the enzyme composition is a fermentation broth composition, optionally subject to some post-production/fermentation processing. In certain embodiments, the enzyme composition is a whole broth formulation.

In some aspects, provided is a saccharification process comprising treating a lignocellulosic biomass material with a composition comprising a polypeptide, wherein the polypeptide has at least about 55% (e.g., at least about 55%, 60%, 65%, 70%, 75%, 80%, 85%, 90%, 91%, 92%, 93%, 94%, 95%, 96%, 97%, 98%, 99%) sequence identity to SEQ ID NO:2, or to the mature sequence of SEQ ID NO:3, and wherein the process results in at least about 50% (e.g., at least about 55%, 60%, 65%, 70%, 75%, 80%, 85%, or 90%) by weight conversion of biomass to fermentable sugars. In some aspects, lignocellulosic biomass material has been subject to one or more pretreatment methods/steps as described herein.

Other aspects and embodiments of the present compositions and methods will be apparent from the foregoing description and following examples.

### EXAMPLES

The following examples are put forth so as to provide those of ordinary skill in the art with a complete disclosure and description of how to make and use the present compositions and methods, and are not intended to limit the scope of what the inventors regard as their inventive compositions and methods nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1

### Cloning of Streptococcus gallolyticus Glycosyl Hydrolase SgaMan1

Streptococcus gallolyticus was selected as a potential source for various glycosyl hydrolases and other enzymes, useful for industrial applications. Genomic DNA for sequencing was obtained by first growing a strain of Streptococcus gallolyticus, UCN34 on LB agar plates at 30° C. for about 24 hours. Cell material was scraped from the plates and used to prepare genomic DNA using phenol/chloroform extraction. The genomic DNA was used for sequencing by BaseClear, NL. Contigs were annotated by BioXpr (Namur, Belgium). The SgaMan1 gene was amplified for subsequent expression cloning.

The SgaMan1 gene was identified from the genomic sequence. The nucleic acid sequence of this gene comprises the polynucleotide sequence of SEQ ID NO:1:

ATGAAAAAAGCATTAGTTGGATCTTTAGCGACCCTAACTGTCGTTGCCG
GTCTCGCATCAGCTCAAGGGGTAAAAGCTGACGAAATCGTTTCAGGACA
GACTTACAAAATCACTGCAAAACATAGTGGTAAAGCTCTTGACGTTGCA
TCAAAAGCAACTTACGCAGGAGCTAACGTGCAACAATGGTCTTATAACG
GCGGCACTAACCAACAATGGAAAGTTGTTGATACAGGAGACGGATACTA
CAAATTAGTATCAGTAAACAGTGGTAAAGTGCTAGACGTAGCAAGTGCA
GCAACTTACAATGGTGCTAACGTACAACAATGGGACGATACTAACGGAA
CATGCCAACGCTGGAAAATCGTAGATACTGGTGACGGTTCTTACAAACT
AATCTCTGCTGTCAGCGGTAAAGCTCTTGACGTTGCAGCAAACTCTAGC
GATGACGGTGCCAACGTACAACAATGGGATGACAACGGTGGTTGGAACC
AAAAATGGGAATTCACAAAAGTAAGTGACAGTGGAAACACTGGTGGTGA
TACTGGTGGCGACACTACTACTCCAGTTTCAACTAGTGGTTTCTACACA
AACGGTACTACACTTTATGACGCAAAAGGTAATCCATTTGTGATGCGCG
GTATCAACCACGCTCACTCTTGGTACCAAGGTTATGAAGATACTGCTAT
TCCTGCCATTGCAAAAACTGGCGCTAACTGTGTTCGCTTAGTTATCACA
GATGGTCAACAATACAGTAAAACTTCACTCTCTGAACTTCAAAAACTTA
TTCAACTTTGTAAAGATAACAAATTAATTGCTATCGTTGAAGTGCATGA
CGCTACTGGTTCAGATAACATTCAAGACTTAGAAAAAGCTGCTGATTAC
TGGATTGAAATGAAGAGTGCTTTGATTGGAAACGAAGCAAACGTCATCC
TTAACATCGCTAACGAATGGGGCGGTGCTTGGGACAGTTCAAACTGGGC
AGCAGGTTACCAACAAGTTATTCCAAAACTTCGTAATGCTGGTATCAAA
AACACACTTATGGTCGATTGTGCTGGTTGGGGACAATACCCACAATCTA
TCGCAGAAGCAGGTCAAAGTGTCGTTCAAGCTGATTCACAACACAACAT
CATGTTCTCTATTCACATGTACGAATATGCTGGTGCATACGGCGCAGTT
CAATCAAACATTGACTCTGCACTTAACGTAGGTGTACCACTTTGTATCG
GTGAATTCGGTATCCGACACACTAACGGCGATGTAGATTACAAGACAAT
CATGAGCTACTCTCAATCTAAGGGAGTTGGTTACCTCGGTTGGTCTTGG
AAAGGTAACGGCGACACTTGGGCTTACCTCGATATCGCACAAGATTGGA
GCGGTTCACAATTAACTGAACAAGGTAACGCTATTATCTATGACCAAAA
TGGTATCAAGAACACATCTAAACGTTGTACAATTTTTGACTAA

The amino acid sequence of the SgaMan1 precursor protein is provided below as SEQ ID NO:2, with the predicted native signal peptide presented in italic and bold letters:

[symbol]

[symbol]

QGVKADEIVSGQTYKITAKHSGKALDV
ASKATYAGANVQQWSYNGGTNQQWKVVDTGDGYYKLVSVNSGKVLDVAS
AATYNGANVQQWDDTNGTCQRWKIVDTGDGSYKLISAVSGKALDVAANS
SDDGANVQQWDDNGGWNQKWEFTKVSDSGNTGGDTGGDTTTPVSTSGFY
TNGTTLYDAKGNPFVMRGINHAHSWYQGYEDTAIPAIAKTGANCVRLVI
TDGQQYSKTSLSELQKLIQLCKDNKLIAIVEVHDATGSDNIQDLEKAAD
YWIEMKSALIGNEANVILNIANEWGGAWDSSNWAAGYQQVIPKLRNAGI
KNTLMVDCAGWGQYPQSIAEAGQSVVQADSQHNIMFSIHMYEYAGAYGA
VQSNIDSALNVGVPLCIGEFGIRHTNGDVDYKTIMSYSQSKGVGYLGWS
WKGNGDTWAYLDIAQDWSGSQLTEQGNAIIYDQNGIKNTSKRCTIFD

The amino acid sequence of the mature SgaMan1 protein is provided below as SEQ ID NO:3:

QGVKADEIVSGQTYKITAKHSGKALDVASKATYAGANVQQWSYNGGTNQ
QWKVVDTGDGYYKLVSVNSGKVLDVASAATYNGANVQQWDDTNGTCQRW
KIVDTGDGSYKLISAVSGKALDVAANSSDDGANVQQWDDNGGWNQKWEF
TKVSDSGNTGGDTGGDTTTPVSTSGFYTNGTTLYDAKGNPFVMRGINHA
HSWYQGYEDTAIPAIAKTGANCVRLVITDGQQYSKTSLSELQKLIQLCK
DNKLIAIVEVHDATGSDNIQDLEKAADYWIEMKSALIGNEANVILNIAN
EWGGAWDSSNWAAGYQQVIPKLRNAGIKNTLMVDCAGWGQYPQSIAEAG
QSVVQADSQHNIMFSIHMYEYAGAYGAVQSNIDSALNVGVPLCIGEFGI
RHTNGDVDYKTIMSYSQSKGVGYLGWSWKGNGDTWAYLDIAQDWSGSQL
TEQGNAIIYDQNGIKNTSKRCTIFD

The polypeptide was predicted to have a signal peptide of 21 amino acid residues in length, using the Signal P 3.0 program (www.cbs.dtu/services/SignalP) set to SignalP-NN system (Emanuelsson et al., Nature Protocols, 2: 953-971, 2007). The presence of a signal sequence suggests that the SgaMan1 polypeptide is a secreted glycosyl hydrolase.

### Example 2

### Expression of Streptococcus gallolyticus Beta-Mannanase SgaMan1 in a Bacillus subtilis Host

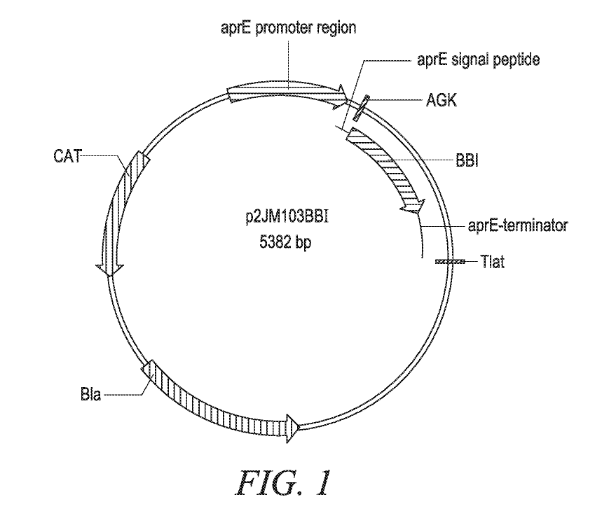

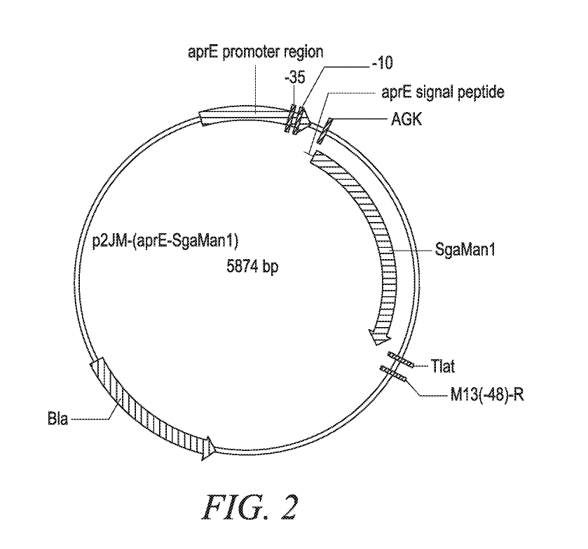

The DNA sequence encoding mature SgaMan1 was synthesized (Generay, Shanghai, P.R. China) with an alternative start codon (GTG) and inserted into a Bacillus subtilis expression vector p2JM103BBI (FIG. 1) (Vogtentanz, Protein Expr. Purif., 55:40-52, 2007). The resulting plasmid was named p2JM-aprE-SgaMan1 (FIG. 2). The plasmid contains an aprE promoter, an aprE signal sequence used to direct target protein secretion in B. subtilis, an oligonucleotide encoding peptide Ala-Gly-Lys to facilitate the secretion of the target enzyme SgaMan1, and the synthetic nucleotide sequence encoding the mature SgaMan1 (SEQ ID NO:3).

The p2JM-aprE-SgaMan1 plasmid (FIG. 2) was then introduced into B. subtilis cells (degUHy32, ΔnprB, Δvpr, Δepr, ΔscoC, ΔwprA, Δmpr, ΔispA, Δbpr) and the thus derived cells were spread on Luria Agar plates supplemented with 5 ppm Chloraphenicol. Colonies were picked and subjected to fermentation in a 250 mL shake flas with an MBD medium (which is a MOPS-based defined medium, supplemented with additional 5 mM CaCl₂).

Following the natural signal peptidase cleavage in the host, the recombinant SgaMan1 polypeptide produced in this manner was predicted to have and had 3 additional amino acids, Ala-Gly-Lys, at its amino-terminus.

The sequence of the SgaMan1 gene was confirmed by DNA sequencing (SEQ ID NO:6). The gene has an alternative start codon (GTG). The oligonucleotide encoding the three residue addition (AGK) is shown by bold and underline:

GTGAGAAGCAAAAAATTGTGGATCAGCTTGTTGTTTGCGTTAACGTTA
ATCTTTACGATGGCGTTCAGCAACATGAGCGCGCAGGCTGCTGGAAAA
CAAGGCGTTAAAGCAGATGAAATTGTTTCAGGCCAGACGTATAAAATC
ACAGCAAAACATTCAGGCAAAGCACTGGATGTTGCAAGCAAAGCAACA
TATGCAGGCGCAAATGTTCAACAGTGGTCATATAATGGCGGAACAAAT
CAACAATGGAAAGTCGTTGATACAGGCGACGGCTATTACAAACTGGTT
TCAGTTAATAGCGGCAAAGTTCTGGATGTCGCATCAGCAGCAACATAC
AATGGCGCTAATGTCCAACAATGGGATGATACAAATGGCACATGCCAG
AGATGGAAAATTGTCGATACAGGTGATGGCAGCTATAAACTGATTTCA
GCAGTTAGCGGAAAAGCGCTTGATGTTGCGGCAAATTCATCAGATGAT
GGCGCAAACGTGCAGCAGTGGGATGACAATGGCGGATGGAATCAAAAA
TGGGAATTTACGAAAGTTAGCGACTCAGGCAATACAGGCGGAGATACG
GGTGGCGATACAACAACACCGGTTTCAACATCAGGCTTTTATACAAAC
GGAACGACACTGTATGATGCAAAAGGCAATCCGTTTGTTATGCGTGGC
ATTAATCATGCACATTCATGGTATCAAGGCTATGAAGATACAGCAATT
CCGGCTATTGCAAAAACAGGCGCTAATTGCGTTAGACTGGTTATTACA
GATGGCCAGCAATATTCAAAAACATCACTGAGCGAACTGCAGAAACTG
ATTCAACTGTGCAAAGACAACAAACTGATCGCAATTGTTGAAGTTCAT
GATGCTACAGGCAGCGATAATATTCAGGATCTGGAAAAAGCAGCGGAC
TATTGGATTGAAATGAAATCAGCACTGATTGGCAATGAAGCGAATGTC
ATTCTGAACATTGCAAATGAATGGGGAGGCGCATGGGATTCATCAAAT
TGGGCAGCAGGCTATCAACAAGTTATTCCGAAACTGAGAAATGCAGGC
ATTAAAAACACACTTATGGTTGATTGCGCAGGCTGGGGACAATATCCG
CAATCAATTGCAGAAGCAGGCCAAAGCGTTGTTCAAGCAGATTCACAA
CATAACATCATGTTTAGCATCCATATGTACGAATACGCAGGCGCATAT
GGCGCAGTTCAATCAAATATTGATAGCGCACTGAATGTTGGCGTTCCG
CTGTGCATTGGCGAATTTGGCATTAGACATACGAATGGTGATGTCGAC
TATAAAACGATCATGAGCTATTCACAATCAAAAGGCGTTGGCTATCTG
GGCTGGTCATGGAAAGGCAACGGCGATACATGGGCATATCTGGATATT
GCACAAGATTGGTCAGGCTCACAACTGACAGAACAAGGCAATGCAATT
ATCTATGATCAGAACGGCATCAAAAATACGTCAAAACGCTGCACGATC
TTTGAT

The amino acid sequence of the full-length SgaMan1 polypeptide expressed from the plasmid p2JM-aprE-SgaMan1 was confirmed and set forth as SEQ ID NO:7, with the signal sequence shown in italics and the three residue addition shown by bold and underline.

MRSKKLWISLLFALTLIFTMAFSNMSAQAAGKQGVKADEIVSGQTYKIT
AKHSGKALDVASKATYAGANVQQWSYNGGTNQQWKVVDTGDGYYKLVSV
NSGKVLDVASAATYNGANVQQWDDTNGTCQRWKIVDTGDGSYKLISAVS
GKALDVAANSSDDGANVQQWDDNGGWNQKWEFTKVSDSGNTGGDTGGDT
TTPVSTSGFYTNGTTLYDAKGNPFVMRGINHAHSWYQGYEDTAIPAIAK
TGANCVRLVITDGQQYSKTSLSELQKLIQLCKDNKLIAIVEVHDATGSD
NIQDLEKAADYWIEMKSALIGNEANVILNIANEWGGAWDSSNWAAGYQQ
VIPKLRNAGIKNTLMVDCAGWGQYPQSIAEAGQSVVQADSQHNIMFSIH
MYEYAGAYGAVQSNIDSALNVGVPLCIGEFGIRHTNGDVDYKTIMSYSQ
SKGVGYLGWSWKGNGDTWAYLDIAQDWSGSQLTEQGNAIIYDQNGIKNT
SKRCTIFD

The amino acid sequence of the SgaMan1 mature polypeptide expressed from the plasmid p2JM-aprE-SgaMan1 was confirmed and set forth as SEQ ID NO:8, with the three residues amino terminal extension based on the predicted cleavage site shown in bold and underline.

AGKQGVKADEIVSGQTYKITAKHSGKALDVASKATYAGANVQQWSYNG
GTNQQWKVVDTGDGYYKLVSVNSGKVLDVASAATYNGANVQQWDDTNG
TCQRWKIVDTGDGSYKLISAVSGKALDVAANSSDDGANVQQWDDNGGW
NQKWEFTKVSDSGNTGGDTGGDTTTPVSTSGFYTNGTTLYDAKGNPFV
MRGINHAHSWYQGYEDTAIPAIAKTGANCVRLVITDGQQYSKTSLSEL
QKLIQLCKDNKLIAIVEVHDATGSDNIQDLEKAADYWIEMKSALIGNE
ANVILNIANEWGGAWDSSNWAAGYQQVIPKLRNAGIKNTLMVDCAGWG
QYPQSIAEAGQSVVQADSQHNIMFSIHMYEYAGAYGAVQSNIDSALNV
GVPLCIGEFGIRHTNGDVDYKTIMSYSQSKGVGYLGWSWKGNGDTWAY
LDIAQDWSGSQLTEQGNAIIYDQNGIKNTSKRCTIFD

After the three terminal extension residues were cleaved, the mature SgaMan1 polypeptide was confirmed to have the sequence of SEQ ID NO:3:

QGVKADEIVSGQTYKITAKHSGKALDVASKATYAGANVQQWSYNGGTN
QQWKVVDTGDGYYKLVSVNSGKVLDVASAATYNGANVQQWDDTNGTCQ
RWKIVDTGDGSYKLISAVSGKALDVAANSSDDGANVQQWDDNGGWNQK
WEFTKVSDSGNTGGDTGGDTTTPVSTSGFYTNGTTLYDAKGNPFVMRG
INHAHSWYQGYEDTAIPAIAKTGANCVRLVITDGQQYSKTSLSELQKL
IQLCKDNKLIAIVEVHDATGSDNIQDLEKAADYWIEMKSALIGNEANV
ILNIANEWGGAWDSSNWAAGYQQVIPKLRNAGIKNTLMVDCAGWGQYP
QSIAEAGQSVVQADSQHNIMFSIHMYEYAGAYGAVQSNIDSALNVGVP
LCIGEFGIRHTNGDVDYKTIMSYSQSKGVGYLGWSWKGNGDTWAYLDI
AQDWSGSQLTEQGNAIIYDQNGIKNTSKRCTIFD

The SgaMan1 polypeptide produced in the Bacillus subtilis host cells, as described above, was secreted into the extracellular culture medium after expression was complete. Accordingly the expression culture medium was filtered and concentrated, and used for protein purification.

### Example 3

### Purification of Beta-Mannanase SgaMan1 from a Culture Medium of Bacillus subtilis

A three-step purification procedure was applied, including an anion exchange, hydrophobic interaction chromatography, and gel filturation. More specifically, about 700 mL crude broth was taken from a shake flask fermentor, concentrated using VIVAfLOW 200 (cutoff 10 kD) and buffer exchanged into 20 mM Tris-HCl, pH 7.5. The broth was then loaded onto a 50-mL Q-Sepharose High Performance column which had been prequilibrated with 20 mM Tris-HCl, pH 7.5 (buffer A). An elution step was then carried out using a linear gradient from 0 to 50% buffer B, which was 20 mM HCl, pH 7.5 with 1 M NaCl, using a total of 3 column volumes, followed with another 3 column volumes of 100% buffer B. The protein of interest, SgaMan1, was detected in the flow-through fraction.

A 3 M ammonium sulfate solution was added to the flow-through fraction to an ultimate concentration of 1 M ammonium sulfate. The thus pretreated fraction was loaded onto a 50-mL Phenyl-Sepharose Fast Flow column equilibrated with 20 mM Tris-HCl, pH 7.5, 1 M ammonium sulfate. A gradient elution was applied, using 3 column volumes of 0-100% buffer A, followed by 3 column volume of 100% buffer A. Relatively pure fractions were selected based on SDS-PAGE. The fractions containing relatively pure enzymes were pooled.

The collected/pooled fractions were concentrated into 10 mL total volume. Then it was loaded onto the HiLoadTM 26/60, Superdex-75 column (1 column volume=320 mL), which had been preequilibrated with 20 mM sodium phosphate buffer, pH 7.0, 0.15 M NaCl. The purities of the fractions were again analyzed using SDS-PAGE.

Results indicated that SgaMan1 was at least 95% if not completely purified.

The pure fractions were pooled and concentrated using an Amicon Ultra-15 device with 10K molecular weight cutoff. The purified sample was stored at −80° C. in 20 mM sodium phosphate buffer (pH 7.0) containing 40% glycerol. Prior to conducting the biochemical analyses below, the frozen purified sample was carefully thawed.

### Example 4

### Prophetic

### Expression of Streptococcus gallolyticus Beta-Mannanase SgaMan1 in a T. reesei Host

The SgaMan1 gene can be amplified from Streptococcus gallolyticus genomic DNA using PCR, with the native signal sequence and a CACC sequence added to the 5′ end of the forward primer for directional Gateway cloning (Invitrogen, Carlsbad, Calif.). Alternatively, a T. reesei cbh1 signal sequence might be employed, substituting for the native signal sequence. The PCR product of the SgaMan1 gene can be purified using a Qiaquick PCR Purification Kit (Qiagen). The purified PCR product can then be cloned into the pENTR/D-TOPO vector, transformed into One Shot® TOP10 Chemically Competent E. coli cells (Invitrogen), and then plated onto LA plates containing 50 ppm kanamycin. Plasmid DNA can then be obtained from the E. coli transformants, using a QIAspin plasmid preparation kit (Qiagen).

The nucleotide sequence of the inserted DNA can then be confirmed as SEQ ID NO:1 using well-known sequencing methods. The pENTR/D-TOPO_SgaMan1 vector including the confirmed SgaMan1 gene sequence can then be recombined with the expression vector pTrex3gM (see, e.g., International Published Patent Application WO 05/001036, FIG. 2), using an LR clonase® reaction (see, protocols by Invitrogen).

The product of the LR clonase® reaction (i.e., the vector pTrex3gM_SgaMan1) can then be transformed into E. coli One Shot® TOP10 Chemically Competent cells (Invitrogen) and plated on LA medium containing 50 ppm carbenicillin. The pTrex3gM vector also contains the Aspergillus tubingensis amdS gene, encoding acetamidase, as a selectable marker for transformation of T. reesei. The pTrex3gM vector further contains a cbh1 promoter and terminator, which flank the SgaMan1 sequence.

Thereafter, about 0.5 to 1 μg of the expression vector pTrex3gM_SgaMan1 (or a fragment amplified by PCR) can be used to transform a T. reesei strain with its major cellulase genes deleted, for example, a six-fold deletion strain as described in, e.g., in International Patent Application Publication No. WO 2010/141779), using the PEG-protoplast method with modifications as described herein.

For protoplast preparation, spores can be grown for 16-24 hours at 24° C. in a Trichoderma Minimal Medium MM, containing 20 g/L glucose, 15 g/L KH₂PO₄, pH 4.5, 5 g/L (NH₄)₂SO₄, 0.6 g/L MgSO₄×7H₂O, 0.6 g/L CaCl₂×2H₂O, 1 mL of 1000× T. reesei Trace elements solution (5 g/L FeSO₄×7H₂O, 1.4 g/L ZnSO₄×7H₂O, 1.6 g/L MnSO₄×H₂O, 3.7 g/L CoCl₂×6H₂O) with shaking at 150 rpm. Germinating spores can then be harvested by centrifugation and treated with 50 mg/mL of Glucanex G200 (Novozymes AG) solution to lyse the fungal cell walls. Further preparation of the protoplasts can be performed in accordance with a method described by Penttilä et al. Gene 61(1987)155-164. The transformation mixture, containing about 1 μg of DNA and at least 1×10⁷ protoplasts in a total volume of 200 μL, can then be treated with 2 mL of 25% PEG solution, diluted with 2 volumes of 1.2 M sorbitol/10 mM Tris, pH7.5, 10 mM CaCl₂, mixed with 3% selective top agarose MM containing 20 mM acetamide. The resulting mixture is then poured onto 2% selective agarose plate containing acetamide. Followed by that, plates are incubated for 7-10 d at 28° C. Single transformants are then transferred onto fresh MM plates containing acetamide. Spores from independent clones are then used to inoculate a fermentation medium in either 96-well microtiter plates or shake flasks.

Secreted protein from the culture broths can be purified, optionally subject to some post-fermentation processing, or can be used directly for saccharification or hydrolyzing mannan-containing lignocellulosic biomass substrates.

### Example 5

### Beta-Mannanase Activity of SgaMan1

The beta-1,4 mannanase activity of SgaMan1 was measured using 0.5% locust bean gum galactomannan from Ceratonia siliqua seeds (Sigma, G0753), and konjac glucomannan (Megazyme P-GLCML) (Bray, Ireland) as substrates.

The assay was performed in a 50 mM sodium acetate buffer, pH 5.0, containing 0.005% Tween-80, whereby the polypeptide and the substrate were incubated at 50° C. for 10 minutes.

The reducing sugar(s) released from the hydrolysis reaction was quantified using a PAHBAH (p-Hydroxy benzoic acid hydrazide) assay as described by Lever (1972) Anal. Biochem. 47:248. A standard curve was prepared using various amounts of mannose as standards, and the specific enzyme activity units were calculated. Specifically one mannanase unit was defined as the amount of enzyme required to generate 1 micromole of mannose reducing sugar equivalents per minute under a given set of conditions.

As measured, the specific activity of the purified SgaMan1 polypeptide was measured to have about 370 units/mg against the Locust bean gum substrate, and about 149 units/mg against the Konjac glucomannan substrate at pH 5.0; and about 799 units/mg against the Locust bean gum substrate, and about 346 units/mg against the Konjac glucomannana at pH 8.2.

### Example 6

### pH Profile of SgaMan1

The pH profile of SgaMan1 was determined using locust bean gum from Ceratonia siliqua seeds (Sigma G0753) as substrate. The enzyme was first diluted in 0.005% Tween-80 to an appropriate concentration based on the dose response curve. The substrate solutions buffered using sodium citrate/sodium phosphate buffers of different pHs were pre-incubated in a thermomixer at 50° C. for 5 minutes.

The activity assays were performed in a sodium citrate/sodium phosphate buffer, having various pH values in a range between pH 2 and pH 9. Assay reactions were initiated by addition of enzymes to the substrate mixture. The mixtures were then incubated at 50° C. for 10 minutes, followed by termination of reactions by transferring 10 μL reaction mixture to a 96-well PCR plate, which were preloaded in each well 100 μL of PAHBAH solutions.

The PCR plate was then incubated at 95° C. for 5 minutes in a Bio-Rad DNA Engine. Then 100 μL of a mixture in each well was transferred to a new 96-well assay plate.

The amount of reducing sugar(s) released from the substrate was determined by measuring the optical density of the reaction mixture following the completion of the reaction as described above at 410 nm in a spectrophotometer. The enzyme activity at each pH was reported as relative activity where the activity at the pH optimum was normalized to 100%.

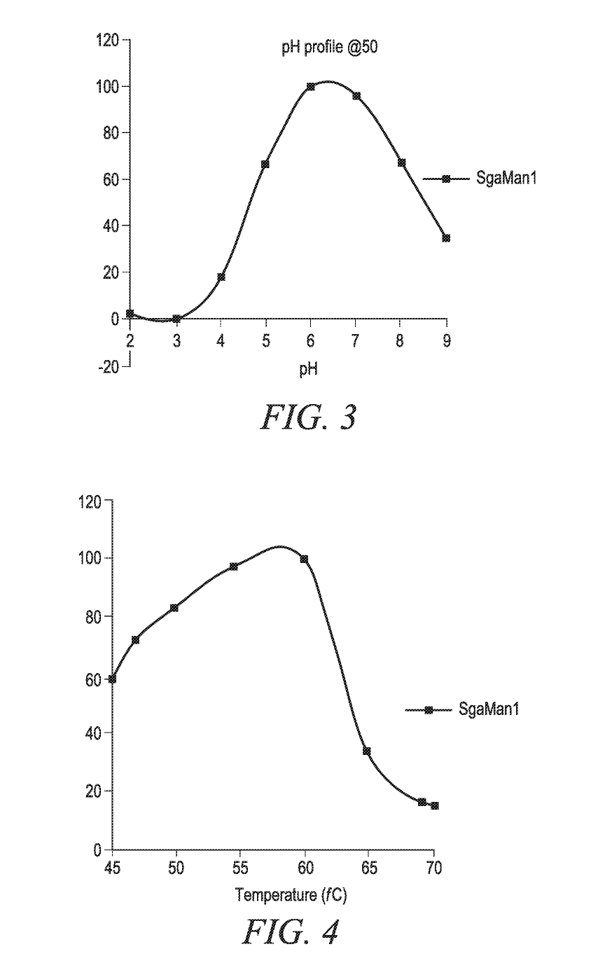

The pH profile of SgaMan1 is shown in FIG. 3. SgaMan1 was found to have an optimum pH at about pH 6.3. The polypeptide was also found to retain greater than 70% of its maximum activity between pH 5.0 and pH 8.0

### Example 7

### Temperature Profile of SgaMan1

The temperature optimum of purified SgaMan1 polypeptide was determined by measuring the beta-mannanase of SgaMan1, at various temperatures between 40° C. and 90° C., in a 50 mM sodium citrate buffer, pH 6.0, for 10 minutes for activity upon the locust bean gum substrate. The activity was reported as relative activity where the activity at the temperature optimum was normalized to 100%. The temperature profile of SgaMan1 is shown in FIG. 4.

SgaMan1 was found to have an optimum temperature of about 58° C. SgaMan1 was also found to retain greater than 70% of its maximum activity between the temperatures of 46° C. and 60° C.

### Example 8

### Thermostability Profile of SgaMan1

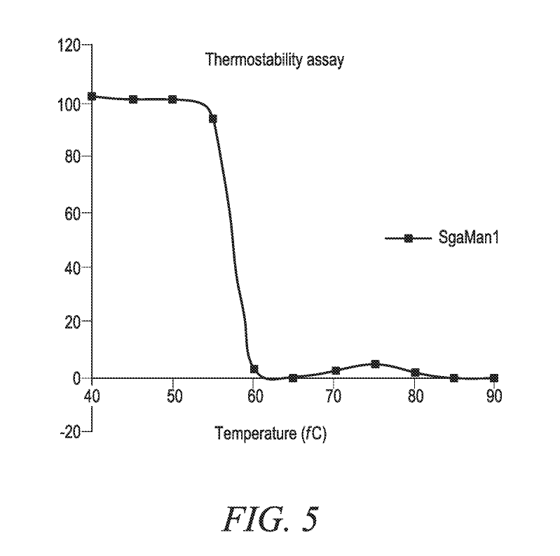

The thermostability of SgaMan1 was determined in a 50 mM sodium citrate buffer, pH 6.0. The enzyme was incubated in a PCR thermal cycler at the desired temperature for 2 hours. The remaining or residual activity of each sample was measured as described in Example 5 above. The activity of a control SgaMan1 sample kept on ice was used to define a 100%-retained activity. The thermostability profile of SgaMan1 is shown in FIG. 5.

SgaMan1 exhibits no detectable activity loss when incubated at a temperature lower than 55° C. for 2 hours. SgaMan1 also retained retained about 50% activity over a 2-hour incubation period at 57° C.

### Example 9

### Hydrolysis Properties and Viscosity Benefits of SgaMan1 as Observed over FPP-27

An alkaline KRAFT-pretreated softwood substrate FPP-27 was obtained from Agence Nationale de la Recherche, France (ARN-05-BIOE-007) through a research project funded by L'Agence Nationale de I'Environmental et de la Maitrise de I'Energie (ADEME 0501 C0099), and a composition analysis was conducted, indicating the following content of the biomass: ˜2.5 wt. % Klason lignin; ˜81.4 wt. % glycan; ˜7.9 wt. % xylan, ˜0.8 wt. % galactan; and ˜6.5 wt. % mannan.

The substrate, in an amount of 1.93 g, at a dry solids loading level of 8.6% and total cellulose loading of 7% was mixed with an Accellerase® TRIO™ sample (which was pre-diluted into the desired concentration, as needed, using 0.05 M sodium citrate buffer, pH 5.0) at 10 mg/g glucan into a reaction mixture as a control. The substrate, in an amount of 1.93 g, at the same dry solids loading level of 8.6% and total cellulose loading of 7%, was mixed with a blended enzyme having 9 mg/g glucan of Accellerase® TRIO™ and 1 mg/g glucan of SgaMan1, or 1 mg/g glucan of XcaMan1, or 1 mg/g SspMan2 in a reaction mixture. The reaction mixtures and the control mixture were adjusted to pH 5 using a 0.1 M sodium citrate buffer. A 5% sodium azide was added to each of the reaction mixtures and control mixture to control microbial growth.

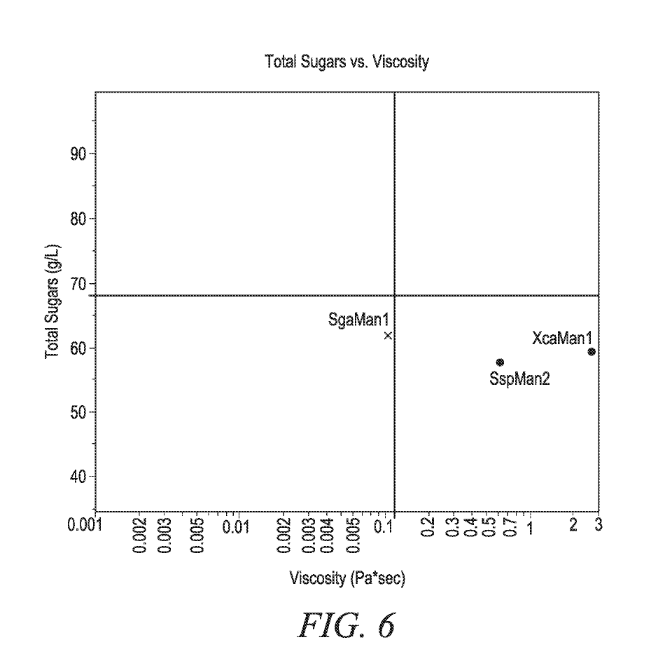

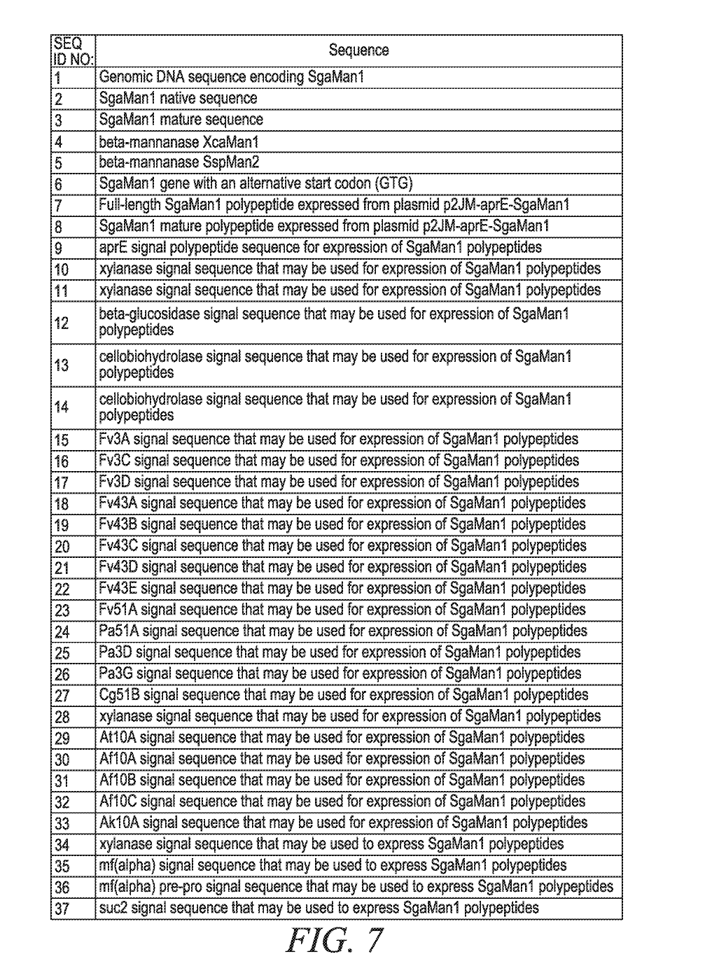

The reaction mixture and the control mixture are then incubated in a New Brunswick Scientific Innova 44 Incubator Shaker at 50° C., with gentle agitation at 200 rpm. After 24 hours, 48 hours, 72 hours, a small sample of about 100 μL was taken from each of the reaction mixture, diluted in 0.9 mL of MilliQ water, followed by filtration through a 0.2 μm filter. The filtrate was then injected into an Waters HPLC, equipped with a Waters 2695 Separation Module, set at a flow rate of 0.6 mL/min, and a mobile phase of MilliQ water degassed with 0.2 μm filter; a Biorad Aminex HPX-87P 300×7.8 mm column, a Phenomenex Security Guard Kit, including a Carbo-Ca 4×3.0 mm security guard cartridge, and a Waters 1260 ELSD detector, set at an operating evaporator and/or nebulizer temperature of 45° C., and gas flow rate of nitrogen at 1.6 SLM. The reaction mixtures as well as the control sample were analyzed for the amount of glucose, xylose, arabinose, and mannose. The results are presented in FIG. 6.

As conducted above, the incubation took place with gentle agitation at a temperature of about 50° C., for at least 72 hours. After at least 72 hours of incubation, the viscosity of each of the resulting mixtures (about 2 to about 3 grams of sample) was determined using the HR-1 rheometer (TA Instruments). A stainless steel 40-mm parallel plate geometry was used. Viscosity evaluation was performed at 23° C. using a sweep shear rate from 50 second⁻¹, decreasing to 1 second⁻¹, over a span of 2 minutes. Based on the stress profiles measured, the Power-law fluid model is applied to determine the viscosity if the hydrolysate in the tested shear rate sweep range.

The SgaMan1 β-mannanase polypeptide, when mixed with Accellerase® TRIO™ in the above-described proportions, imparted a substantial and clear viscosity reduction benefit, as compared to the control samples. The viscosity benefits are presented in a comparison plot of FIG. 6.

### Example 10

### Prophetic

### Hydrolysis and Viscosity Benefits of SgaMan1 as Observed over SPORL-Pretreated Softwood Substrate & Acid-Pretreated Whole Hydrolysate Corn Ctover (whPCS)

A SPORL-preatreated softwood substrate, which has been determined by a composition analysis to contain the following: ˜32.4 wt. % klason lignin; ˜49.4 wt. % glucan; ˜3.4 wt. % xylan; and ˜4.6 wt. % mannan can be used to further indicate hydrolysis benefit and viscosity benefits of SgaMan1. As a control substrate, an acid-pretreated whole hydrolysate corn stover (whPCS) (see, e.g., www.nrel.gov/ docs/fy11osti/47764.pdf), which does not contain any GGM or GM, but contains ˜33.8 wt. % glucan, no xylan, and ˜2.2 wt. % galactan, can be used.

An amount of 1.93 g of such a substrate (including, for example the FPP-27 substrate or the SPORL-pretreated softwood substrate, and the control whPCS substrate), at a dry solids loading level of 8.6% and a total glucan loading of 7.0%, can then be mixed with 10 mg/g glucan of Accellerase® TRIO™ as a control mixture, and with 1 mg/g glucan of SgaMan1 plus 9 mg/g glucan of Accellerase® TRIO™ in a reaction mixture. The reaction mixture and the control mixture are then adjusted to pH 5.0 using a 0.1 M sodium citrate buffer, and incubation can take place with gentle agitation at a temperature of about 50° C., for at least 16 hours.

After at least 72 hours of incubation, the viscosity of each of the resulting mixtures (about 1.2-1.75 grams of sample) can be determined using the HR-1 rheometer (TA Instruments). A stainless steel 40-mm parallel plate geometry is used. Viscosity evaluation is performed at 23° C. using a sweep shear rate from 50 second⁻¹ to 1 second⁻¹. The SgaMan1 β-mannanase polypeptide, when mixed with Accellerase® TRIO™ in the above-described proportions, imparts a substantial and clear viscosity reduction benefit as compared to when the control substrate whPCS is used.

Although the foregoing compositions and methods has been described in some detail by way of illustration and example for purposes of clarity of understanding, it is readily apparent to those of ordinary skill in the art in light of the teachings herein that certain changes and modifications may be made thereto without departing from the spirit or scope of the appended claims.

Accordingly, the preceding merely illustrates the principles of the present compositions and methods. It will be appreciated that those skilled in the art will be able to devise various arrangements which, although not explicitly described or shown herein, embody the principles of the present compositions and methods and are included within its spirit and scope. Furthermore, all examples and conditional language recited herein are principally intended to aid the reader in understanding the principles of the present compositions and methods and the concepts contributed by the inventors to furthering the art, and are to be construed as being without limitation to such specifically recited examples and conditions. Moreover, all statements herein reciting principles, aspects, and embodiments of the present compositions and methods as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present compositions and methods, therefore, is not intended to be limited to the exemplary embodiments shown and described herein.

1-12. (canceled)

13. An enzyme composition comprising a recombinant polypeptide comprising an amino acid sequence that is at least 55% identical to the amino acid sequence of SEQ ID NO:2 or SEQ ID NO:3, wherein the polypeptide has beta-mannanase activity and further comprising one or more cellulases.

14. The enzyme composition of claim 13, wherein the one or more cellulases are selected from one or more beta-glucosidases, one or more cellobiohydrolases, and one or more endoglucanases.

15. An enzyme composition comprising a recombinant polypeptide comprising an amino acid sequence that is at least 55% identical to the amino acid sequence of SEQ ID NO:2 or SEQ ID NO:3, wherein the polypeptide has beta-mannanase activity and further comprising one or more other hemicellulases.

16. The enzyme composition of claim 15, wherein the one or more other hemicellulases are selected from one or more other beta-mannanases, one or more one or more xylanases, one or more beta-xylosidases, and one or more L-arabinofuranosidases.

17-20. (canceled)

21. A host cell comprising an expression vector comprising a nucleic acid in operable combination with a regulatory sequence, the nucleic acid encoding a recombinant polypeptide comprising an amino acid sequence that is at least 55% identical to the amino acid sequence of SEQ ID NO:2 or SEQ ID NO:3, wherein the polypeptide has beta-mannanase activity.

22. The host cell of claim 21, wherein the host cell is a bacterial cell or a fungal cell.

23. A composition comprising the host cell of claim 21 and a culture medium.

24. A method of producing a beta-mannanase, comprising:
culturing the host cell of claim 21 in a culture medium, under suitable conditions to produce the beta-mannanase.

25. A composition comprising the beta-mannanase produced in accordance with the method of claim 24 in supernatant of the culture medium.

26. A method for hydrolyzing a lignocellulosic biomass substrate, comprising: contacting the lignocellulosic biomass substrate with a recombinant polypeptide comprising an amino acid sequence that is at least 55% identical to the amino acid sequence of SEQ ID NO:2 or SEQ ID NO:3, wherein the polypeptide has beta-mannanase activity to yield glucose and other sugars.

27. The method of claim 26, wherein the lignocellulosic biomass substrate comprises up to about 20 wt. %, up to about 15%, or up to about 10 wt. % of galactoglucomannan and/or glucomannan.

28. A composition comprising a recombinant polypeptide comprising an amino acid sequence that is at least 55% identical to the amino acid sequence of SEQ ID NO:2 or SEQ ID NO:3, wherein the polypeptide has beta-mannanase activity, and a lignocellulosic biomass substrate.

29. The composition of claim 28, wherein the lignocellulosic biomass substrate comprises up to about 20 wt. %, or up to about 15 wt. %, or up to about 10 wt. % of galactoglucomannan and/or glucomannan.
